(19) United States
(12) Patent Application Publication
(10) Pub. No.: US 2024/0075225 A1
(43) Pub. Date: Mar. 7, 2024

(54) SYSTEMS AND METHODS FOR LEAK DETECTION IN A RESPIRATORY THERAPY SYSTEM

(71) Applicant: ResMed Sensor Technologies Limited, Dublin (IE)

(72) Inventors: Roxana TIRON, Dublin (IE); Graeme Alexander LYON, Dublin (IE)

(21) Appl. No.: 18/273,662

(22) PCT Filed: Jan. 28, 2022
(86) PCT No.: PCT/IB2022/050742

§ 371 Date: Jul. 21, 2023

Related U.S. Application Data
(60) Provisional application No. 63/143,743, filed on Jan. 29, 2021.

(51) Int. Cl. A61M 16/00; A61M 16/08; A61M 16/16; G16H 40/40

(52) U.S. Cl. CPC A61M 16/0051; A61M 16/0066; A61M 16/024; A61M 16/0875; A61M 16/16; G16H 40/40; A61M 2016/0033; A61M 2205/15; A61M 2205/3375; A61M 2205/505; A61M 2205/581

(57) ABSTRACT

Various implementations of the present disclosure are directed to systems and methods for leak detection in a respiratory therapy system using acoustic data. According to some implementations, a method includes receiving acoustic data associated with airflow caused by operation of a respiratory therapy system during a sleep session of a user. The method also includes analyzing at least a portion of the acoustic data to determine a value of a parameter associated with the at least a portion of the acoustic data. The method further includes determining an occurrence of a leak during the sleep session in response to the determined value of the parameter satisfying a condition.

### CROSS REFERENCE TO RELATED APPLICATIONS

The present application claims the benefit of U.S. Provisional Patent Application No. 63/143,743 filed Jan. 29, 2021, and titled “SYSTEMS AND METHODS FOR LEAK DETECTION IN A RESPIRATORY THERAPY SYSTEM,” the disclosure of which is hereby incorporated by reference in its entirety.

### TECHNICAL FIELD

The present disclosure relates generally to systems and methods for leak detection in a respiratory therapy system, and more particularly, to systems and methods for leak detection in a respiratory therapy system using acoustic data.

### BACKGROUND

Many individuals suffer from sleep-related and/or respiratory disorders such as, for example, Periodic Limb Movement Disorder (PLMD), Restless Leg Syndrome (RLS), Sleep-Disordered Breathing (SDB), Obstructive Sleep Apnea (OSA), apneas, Cheyne-Stokes Respiration (CSR), respiratory insufficiency, Obesity Hyperventilation Syndrome (OHS), Chronic Obstructive Pulmonary Disease (COPD), Neuromuscular Disease (NMD), and chest wall disorders. These disorders are often treated using a respiratory therapy system. The respiratory therapy system may have or develop leaks in airflow at different locations during use. Estimation of such leaks is a function of the impedance of airflow within portions of the respiratory therapy system. However, errors in determining the impedance of the airflow, for example, due to manufacturing errors or configuration issues during use, often lead to inaccurate estimation of such leaks. Accordingly, a better method of detecting and measuring such leaks is desirable for operation of the respiratory therapy system.

### SUMMARY

According to some implementations of the present disclosure, a method includes receiving acoustic data associated with airflow caused by operation of a respiratory therapy system during a sleep session of a user. The method also includes analyzing at least a portion of the acoustic data to determine a value of a parameter associated with the at least a portion of the acoustic data. The method further includes determining an occurrence of a leak during the sleep session in response to the determined value of the parameter satisfying a condition.

According to some implementations of the present disclosure, a system includes a respiratory therapy device, a user interface, a conduit, a microphone, a memory, and a control system. The respiratory therapy device is configured to generate a flow of pressurized air. The user interface is configured to aid in delivery of the flow of pressurized air to a user. The conduit is configured to connect the respiratory therapy device and the user interface. The memory stores machine-readable instructions. The control system includes one or more processors configured to execute the machine-readable instructions to generate, using the microphone, acoustic data associated with the flow of the pressurized air during a sleep session of the user. The control system is also configured to analyze at least a portion of the generated acoustic data to determine a value of a parameter associated with the generated acoustic data. The control system is further configured to determine an occurrence of a leak during the sleep session in response to the determined value of the parameter satisfying a condition.

The above summary is not intended to represent each implementation or every aspect of the present disclosure. Additional features and benefits of the present disclosure are apparent from the detailed description and figures set forth below.

### BRIEF DESCRIPTION OF THE DRAWINGS

FIG. 1 is a functional block diagram of a respiratory therapy system, according to some implementations of the present disclosure.

FIG. 2 is a perspective view of at least a portion of the system of FIG. 1, a user, and a bed partner, according to some implementations of the present disclosure.

FIG. 3A is a perspective view of a microphone configured to detect a leak in the system of FIG. 1, according to some implementations of the present disclosure.

FIG. 3B is a schematic representation of three microphones configured to detect presence and location of a leak in the system of FIG. 1, according to some implementations of the present disclosure.

FIG. 4 illustrates an exemplary timeline for a sleep session, according to some implementations of the present disclosure.

FIG. 5A illustrates a comparative graphical representation of mask pressure (i.e. pressure in the user interface of the respiratory therapy system of FIG. 1), flow rate, leak rate, standard deviation of acoustic intensity, and maximum value of acoustic intensity over a time period during which leaks occur in the respiratory therapy system of FIG. 1, according to some implementations of the present disclosure.

FIG. 5B illustrates the peaks at fundamental frequency and harmonic frequencies representing high-intensity whistle-like sounds on the acoustic spectra that can be used to estimate an amount of leak, according to some implementations of the present disclosure.

FIG. 6A illustrates a graphical representation of acoustic power levels over a five-minute time period of no leak and a five-minute time period of leak in the respiratory therapy system of FIG. 1, according to some implementations of the present disclosure.

FIG. 6B illustrates a comparative graphical representation of flow rate, mask pressure (i.e. pressure in the user interface of the respiratory therapy system of FIG. 1), and leak rate over the five-minute time periods of leak and no leak of FIG. 6A in the respiratory therapy system of FIG. 1, according to some implementations of the present disclosure.

FIG. 7A illustrates a graphical representation of acoustic power levels over a time period during which leaks occur in the respiratory therapy system of FIG. 1, according to some implementations of the present disclosure.

FIG. 7B illustrates a comparative graphical representation of flow rate, mask pressure (i.e. pressure in the user interface of the respiratory therapy system of FIG. 1), and leak rate over the time period of FIG. 7A during which leaks occur in the respiratory therapy system of FIG. 1, according to some implementations of the present disclosure.

FIG. 8A illustrates plotted Cartesian coordinates representing average device pressure and average total flow rate, according to some implementations of the present disclosure.

FIG. 8B illustrates a fitted characteristic curve over the plotted Cartesian coordinates of FIG. 8A, according to some implementations of the present disclosure.

FIG. 9 illustrates an intentional leak characteristic curve of a respiratory therapy system associated with a user, according to some implementations of the present disclosure.

FIG. 10 illustrates a flow diagram for a method of detecting a leak in the respiratory therapy system, according to some implementations of the present disclosure.

While the present disclosure is susceptible to various modifications and alternative forms, specific implementations and embodiments thereof have been shown by way of example in the drawings and will herein be described in detail. It should be understood, however, that it is not intended to limit the present disclosure to the particular forms disclosed, but on the contrary, the present disclosure is to cover all modifications, equivalents, and alternatives falling within the spirit and scope of the present disclosure as defined by the appended claims.

### DETAILED DESCRIPTION

Many individuals suffer from sleep-related and/or respiratory disorders. Examples of sleep-related and/or respiratory disorders include Periodic Limb Movement Disorder (PLMD), Restless Leg Syndrome (RLS), Sleep-Disordered Breathing (SDB), Obstructive Sleep Apnea (OSA), apneas, Cheyne-Stokes Respiration (CSR), respiratory insufficiency, Obesity Hyperventilation Syndrome (OHS), Chronic Obstructive Pulmonary Disease (COPD), Neuromuscular Disease (NMD), and chest wall disorders.

Obstructive Sleep Apnea (OSA) is a form of Sleep Disordered Breathing (SDB), and is characterized by events including occlusion or obstruction of the upper air passage during sleep resulting from a combination of an abnormally small upper airway and the normal loss of muscle tone in the region of the tongue, soft palate and posterior oropharyngeal wall. More generally, an apnea generally refers to the cessation of breathing caused by blockage of the air (Obstructive Sleep Apnea) or the stopping of the breathing function (often referred to as central apnea). Typically, the individual will stop breathing for between about 15 seconds and about 30 seconds during an obstructive sleep apnea event.

Other types of apneas include hypopnea, hyperpnea, and hypercapnia. Hypopnea is generally characterized by slow or shallow breathing caused by a narrowed airway, as opposed to a blocked airway. Hyperpnea is generally characterized by an increase depth and/or rate of breathing. Hypercapnia is generally characterized by elevated or excessive carbon dioxide in the bloodstream, typically caused by inadequate respiration.

Cheyne-Stokes Respiration (CSR) is another form of sleep disordered breathing. CSR is a disorder of a patient's respiratory controller in which there are rhythmic alternating periods of waxing and waning ventilation known as CSR cycles. CSR is characterized by repetitive de-oxygenation and re-oxygenation of the arterial blood.

Obesity Hyperventilation Syndrome (OHS) is defined as the combination of severe obesity and awake chronic hypercapnia, in the absence of other known causes for hypoventilation. Symptoms include dyspnea, morning headache and excessive daytime sleepiness.

Chronic Obstructive Pulmonary Disease (COPD) encompasses any of a group of lower airway diseases that have certain characteristics in common, such as increased resistance to air movement, extended expiratory phase of respiration, and loss of the normal elasticity of the lung.

Neuromuscular Disease (NMD) encompasses many diseases and ailments that impair the functioning of the muscles either directly via intrinsic muscle pathology, or indirectly via nerve pathology. Chest wall disorders are a group of thoracic deformities that result in inefficient coupling between the respiratory muscles and the thoracic cage.

These and other disorders are characterized by particular events (e.g., snoring, an apnea, a hypopnea, a restless leg, a sleeping disorder, choking, an increased heart rate, labored breathing, an asthma attack, an epileptic episode, a seizure, or any combination thereof) that occur when the individual is sleeping.

The Apnea-Hypopnea Index (AHI) is an index used to indicate the severity of sleep apnea during a sleep session. The AHI is calculated by dividing the number of apnea and/or hypopnea events experienced by the user during the sleep session by the total number of hours of sleep in the sleep session. The event can be, for example, a pause in breathing that lasts for at least 10 seconds. An AHI that is less than 5 is considered normal. An AHI that is greater than or equal to 5, but less than 15 is considered indicative of mild sleep apnea. An AHI that is greater than or equal to 15, but less than 30 is considered indicative of moderate sleep apnea. An AHI that is greater than or equal to 30 is considered indicative of severe sleep apnea. In children, an AHI that is greater than 1 is considered abnormal. Sleep apnea can be considered “controlled” when the AHI is normal, or when the AHI is normal or mild. The AHI can also be used in combination with oxygen desaturation levels to indicate the severity of Obstructive Sleep Apnea.

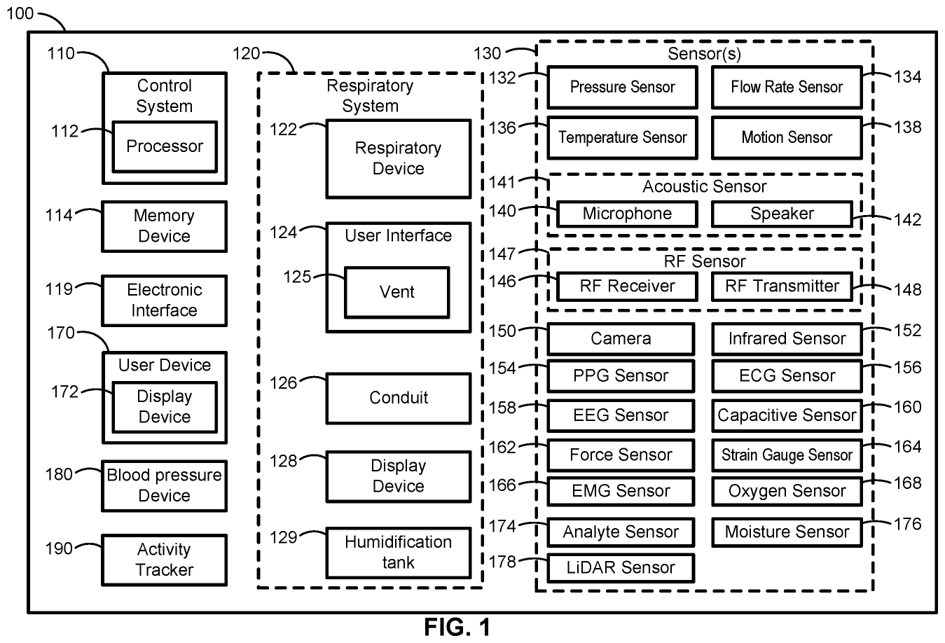

Referring to FIG. 1, a system 100, according to some implementations of the present disclosure, is illustrated. The system 100 includes a control system 110, a memory device 114, an electronic interface 119, one or more sensors 130, and one or more user devices 170. In some implementations, the system 100 further optionally includes a respiratory therapy system 120 (that includes a respiratory therapy device 122), a blood pressure device 180, an activity tracker 190, or any combination thereof.

The control system 110 includes one or more processors 112 (hereinafter, processor 112). The control system 110 is generally used to control (e.g., actuate) the various components of the system 100 and/or analyze data obtained and/or generated by the components of the system 100. The processor 112 can be a general or special purpose processor or microprocessor. While one processor 112 is shown in FIG. 1, the control system 110 can include any suitable number of processors (e.g., one processor, two processors, five processors, ten processors, etc.) that can be in a single housing, or located remotely from each other. The control system 110 can be coupled to and/or positioned within, for example, a housing of the user device 170, and/or within a housing of one or more of the sensors 130. The control system 110 can be centralized (within one such housing) or decentralized (within two or more of such housings, which are physically distinct). In such implementations including two or more housings containing the control system 110, such housings can be located proximately and/or remotely from each other.

The memory device 114 stores machine-readable instructions that are executable by the processor 112 of the control system 110. The memory device 114 can be any suitable computer readable storage device or media, such as, for example, a random or serial access memory device, a hard drive, a solid state drive, a flash memory device, etc. While one memory device 114 is shown in FIG. 1, the system 100 can include any suitable number of memory devices 114 (e.g., one memory device, two memory devices, five memory devices, ten memory devices, etc.). The memory device 114 can be coupled to and/or positioned within a housing of the respiratory therapy device 122, within a housing of the user device 170, within a housing of one or more of the sensors 130, or any combination thereof. Like the control system 110, the memory device 114 can be centralized (within one such housing) or decentralized (within two or more of such housings, which are physically distinct).

In some implementations, the memory device 114 (FIG. 1) stores a user profile associated with a user. The user profile can include, for example, demographic information associated with the user, biometric information associated with the user, medical information associated with the user, self-reported user feedback, sleep parameters associated with the user (e.g., sleep-related parameters recorded from one or more earlier sleep sessions), or any combination thereof. The demographic information can include, for example, information indicative of an age of the user, a gender of the user, a race of the user, a family history of insomnia or sleep apnea, an employment status of the user, an educational status of the user, a socioeconomic status of the user, or any combination thereof. The medical information can include, for example, information indicative of one or more medical conditions associated with the user, medication usage by the user, or both. The medical information data can further include a multiple sleep latency test (MSLT) result or score and/or a Pittsburgh Sleep Quality Index (PSQI) score or value. The self-reported user feedback can include information indicative of a self-reported subjective sleep score (e.g., poor, average, excellent), a self-reported subjective stress level of the user, a self-reported subjective fatigue level of the user, a self-reported subjective health status of the user, a recent life event experienced by the user, or any combination thereof.

The electronic interface 119 is configured to receive data (e.g., physiological data and/or acoustic data) from the one or more sensors 130 such that the data can be stored in the memory device 114 and/or analyzed by the processor 112 of the control system 110. The electronic interface 119 can communicate with the one or more sensors 130 using a wired connection or a wireless connection (e.g., using an RF communication protocol, a WiFi communication protocol, a Bluetooth communication protocol, over a cellular network, etc.). The electronic interface 119 can include an antenna, a receiver (e.g., an RF receiver), a transmitter (e.g., an RF transmitter), a transceiver, or any combination thereof. The electronic interface 119 can also include one more processors and/or one more memory devices that are the same as, or similar to, the processor 112 and the memory device 114 described herein. In some implementations, the electronic interface 119 is coupled to or integrated in the user device 170. In other implementations, the electronic interface 119 is coupled to or integrated (e.g., in a housing) with the control system 110 and/or the memory device 114.

As noted above, in some implementations, the system 100 optionally includes a respiratory therapy system 120 (also referred to as a respiratory therapy system 120). The respiratory therapy system 120 can include a respiratory pressure therapy device 122 (referred to herein as respiratory therapy device 122), a user interface 124, a conduit 126 (also referred to as a tube or an air circuit), a display device 128, a humidification tank 129, or any combination thereof. In some implementations, the control system 110, the memory device 114, the display device 128, one or more of the sensors 130, and the humidification tank 129 are part of the respiratory therapy device 122. Respiratory pressure therapy refers to the application of a supply of air to an entrance of the user's airways at a controlled target pressure that is nominally positive with respect to atmosphere throughout the user's breathing cycle (e.g., in contrast to negative pressure therapies such as the tank ventilator or cuirass). The respiratory therapy system 120 is generally used to treat individuals suffering from one or more sleep-related respiratory disorders (e.g., obstructive sleep apnea, central sleep apnea, or mixed sleep apnea).

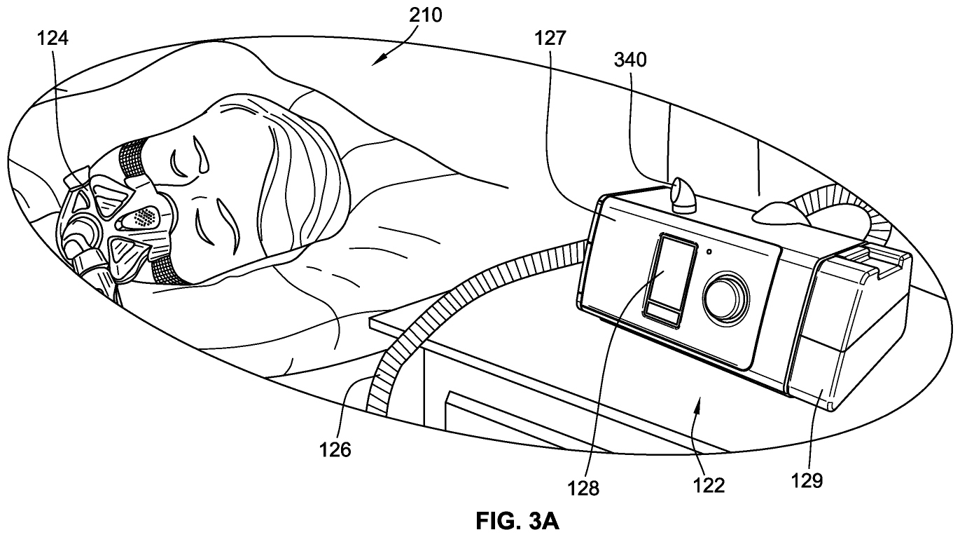

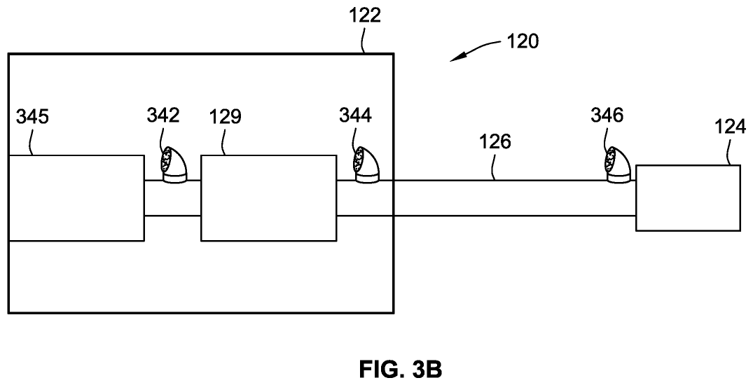

The respiratory therapy device 122 has a blower motor 345 (FIG. 3B) that is generally used to generate pressurized air that is delivered to the user (e.g., using one or more motors that drive one or more compressors). In some implementations, the respiratory therapy device 122 generates continuous constant air pressure that is delivered to the user. In other implementations, the respiratory therapy device 122 generates two or more predetermined pressures (e.g., a first predetermined air pressure and a second predetermined air pressure). In still other implementations, the respiratory therapy device 122 is configured to generate a variety of different air pressures within a predetermined range. For example, the respiratory therapy device 122 can deliver at least about 6 cm H₂O, at least about 10 cm H₂O, at least about 20 cm H₂O, between about 6 cm H₂O and about 10 cm H₂O, between about 7 cm H₂O and about 12 cm H₂O, etc. The respiratory therapy device 122 can also deliver pressurized air at a predetermined flow rate between, for example, about −20 L/min and about 150 L/min, while maintaining a positive pressure (relative to the ambient pressure).

The user interface 124 engages a portion of the user's face and delivers pressurized air from the respiratory therapy device 122 to the user's airway to aid in preventing the airway from narrowing and/or collapsing during sleep. This may also increase the user's oxygen intake during sleep. Depending upon the therapy to be applied, the user interface 124 may form a seal, for example, with a region or portion of the user's face, to facilitate the delivery of air at a pressure at sufficient variance with ambient pressure to effect therapy, for example, at a positive pressure of about 10 cm H₂O relative to ambient pressure. For other forms of therapy, such as the delivery of oxygen, the user interface may not include a seal sufficient to facilitate delivery to the airways of a supply of gas at a positive pressure of about 10 cm H₂O.

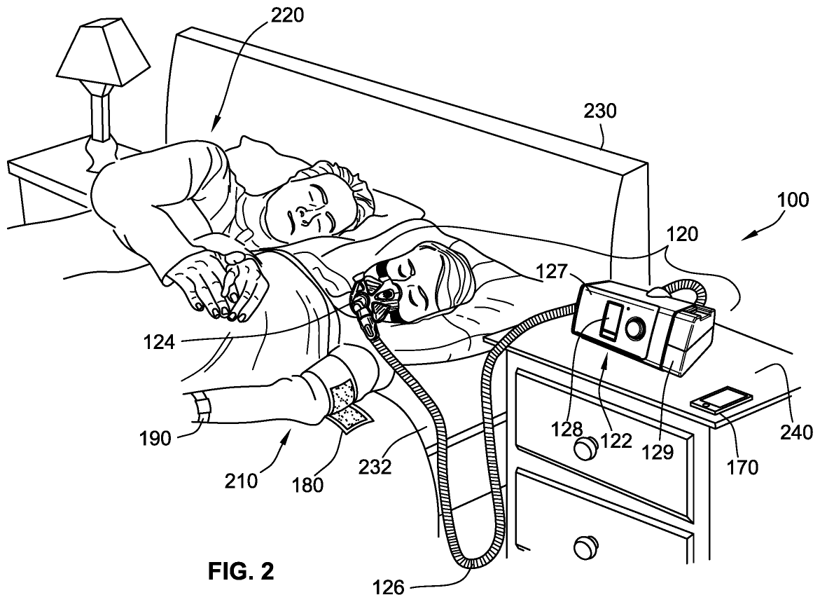

As shown in FIG. 2, in some implementations, the user interface 124 is a facial mask that covers the nose and mouth of the user 210. Alternatively, the user interface 124 can be a nasal mask that provides air to the nose of the user 210 or a nasal pillow mask that delivers air directly to the nostrils of the user 210. The user interface 124 can include a plurality of straps (e.g., including hook and loop fasteners) for positioning and/or stabilizing the interface on a portion of the user 210 (e.g., the face) and a conformal cushion (e.g., silicone, plastic, foam, etc.) that aids in providing an air-tight seal between the user interface 124 and the user 210. The user interface 124 can also include one or more vents 125 for permitting the escape of carbon dioxide and other gases exhaled by the user 210. In other implementations, the user interface 124 includes a mouthpiece (e.g., a night guard mouthpiece molded to conform to the teeth of the user 210, a mandibular repositioning device, etc.).

The conduit 126 (also referred to as an air circuit or tube) allows the flow of air between two components of a respiratory therapy system 120, such as the respiratory therapy device 122 and the user interface 124. In some implementations, there can be separate limbs of the conduit 126 for inhalation and exhalation. In other implementations, a single limb conduit is used for both inhalation and exhalation.

One or more of the respiratory therapy device 122, the user interface 124, the conduit 126, the display device 128, and the humidification tank 129 can contain one or more sensors (e.g., a pressure sensor, a flow rate sensor, or more generally any of the other sensors 130 described herein). These one or more sensors can be used, for example, to measure the air pressure and/or flow rate of pressurized air supplied by the respiratory therapy device 122.

The display device 128 is generally used to display image(s) including still images, video images, or both and/or information regarding the respiratory therapy device 122. For example, the display device 128 can provide information regarding the status of the respiratory therapy device 122 (e.g., whether the respiratory therapy device 122 is on/off, the pressure of the air being delivered by the respiratory therapy device 122, the temperature of the air being delivered by the respiratory therapy device 122, etc.) and/or other information (e.g., a sleep score, the current date/time, personal information for the user 210, etc.). In some implementations, the display device 128 acts as a human-machine interface (HMI) that includes a graphic user interface (GUI) configured to display the image(s) as an input interface. The display device 128 can be an LED display, an OLED display, an LCD display, or the like. The input interface can be, for example, a touchscreen or touch-sensitive substrate, a mouse, a keyboard, or any sensor system configured to sense inputs made by a human user interacting with the respiratory therapy device 122.

The humidification tank 129 is coupled to or integrated in the respiratory therapy device 122 and includes a reservoir of water that can be used to humidify the pressurized air delivered from the respiratory therapy device 122. The respiratory therapy device 122 can include one or more vents (not shown) and a heater to heat the water in the humidification tank 129 in order to humidify the pressurized air provided to the user 210. Additionally, in some implementations, the conduit 126 can also include a heating element (e.g., coupled to and/or imbedded in the conduit 126) that heats the pressurized air delivered to the user 210. In some implementations, the humidification tank 129 may not include the reservoir of water and thus waterless.

The respiratory therapy system 120 can be used, for example, as a ventilator or as a positive airway pressure (PAP) system, such as a continuous positive airway pressure (CPAP) system, an automatic positive airway pressure system (APAP), a bi-level or variable positive airway pressure system (BPAP or VPAP), or any combination thereof. The CPAP system delivers a predetermined amount of pressurized air (e.g., determined by a sleep physician) to the user 210. The APAP system automatically varies the pressurized air delivered to the user 210 based on, for example, respiration data associated with the user 210. The BPAP or VPAP system is configured to deliver a first predetermined pressure (e.g., an inspiratory positive airway pressure or IPAP) and a second predetermined pressure (e.g., an expiratory positive airway pressure or EPAP) that is lower than the first predetermined pressure.

Referring again to FIG. 2, a portion of the system 100 (FIG. 1), according to some implementations, is illustrated. The user 210 of the respiratory therapy system 120 and a bed partner 220 are located on a bed 230 and laying on a mattress 232. The user interface 124 (e.g., a full facial mask) can be worn by the user 210 during a sleep session. The user interface 124 is fluidly coupled and/or connected to the respiratory therapy device 122 via the conduit 126. In turn, the respiratory therapy device 122 delivers pressurized air to the user 210 via the conduit 126 and the user interface 124 to increase the air pressure in the throat of the user 210 to aid in preventing the airway from closing and/or narrowing during sleep. The respiratory therapy device 122 can be positioned on a nightstand 240 that is directly adjacent to the bed 230 as shown in FIG. 2, or more generally, on any surface or structure that is generally adjacent to the bed 230 and/or the user 210.

Referring to back to FIG. 1, the one or more sensors 130 of the system 100 include a pressure sensor 132, a flow rate sensor 134, temperature sensor 136, a motion sensor 138, a microphone 140, a speaker 142, a radio-frequency (RF) receiver 146, a RF transmitter 148, a camera 150, an infrared sensor 152, a photoplethysmogram (PPG) sensor 154, an electrocardiogram (ECG) sensor 156, an electroencephalography (EEG) sensor 158, a capacitive sensor 160, a force sensor 162, a strain gauge sensor 164, an electromyography (EMG) sensor 166, an oxygen sensor 168, an analyte sensor 174, a moisture sensor 176, a LiDAR sensor 178, or any combination thereof. Generally, each of the one or sensors 130 are configured to output sensor data that is received and stored in the memory device 114 or one or more other memory devices. While the one or more sensors 130 are shown and described as including each of the different sensors noted above, more generally, the one or more sensors 130 can include any combination and any number of each of the sensors described and/or shown herein.

The one or more sensors 130 can be used to generate, for example, physiological data, acoustic data, or both. Physiological data generated by one or more of the sensors 130 can be used by the control system 110 to determine a sleep-wake signal associated with the user 210 during a sleep session and one or more sleep-related parameters. The sleep-wake signal can be indicative of one or more sleep states, including wakefulness, relaxed wakefulness, micro-awakenings, a rapid eye movement (REM) stage, a first non-REM stage (often referred to as “N1”), a second non-REM stage (often referred to as “N2”), a third non-REM stage (often referred to as “N3”), or any combination thereof. The sleep-wake signal can also be timestamped to indicate a time that the user 210 enters the bed 230, a time that the user 210 exits the bed 230, a time that the user 210 attempts to fall asleep, etc. The sleep-wake signal can be measured by the sensor(s) 130 during the sleep session at a predetermined sampling rate, such as, for example, one sample per second, one sample per 30 seconds, one sample per minute, etc. Examples of the one or more sleep-related parameters that can be determined for the user 210 during the sleep session based on the sleep-wake signal include a total time in bed, a total sleep time, a sleep onset latency, a wake-after-sleep-onset parameter, a sleep efficiency, a fragmentation index, or any combination thereof.

Physiological data and/or acoustic data generated by the one or more sensors 130 can also be used to determine a respiration signal associated with the user 210 during a sleep session. The respiration signal is generally indicative of respiration or breathing of the user 210 during the sleep session. The respiration signal can be indicative of, for example, a respiration rate, a respiration rate variability, an inspiration amplitude, an expiration amplitude, an inspiration-expiration ratio, a number of events per hour, a pattern of events, pressure settings of the respiratory therapy device 122, or any combination thereof. The event(s) can include snoring, apneas, central apneas, obstructive apneas, mixed apneas, hypopneas, a mask leak (e.g., from the user interface 124), a restless leg, a sleeping disorder, choking, an increased heart rate, labored breathing, an asthma attack, an epileptic episode, a seizure, or any combination thereof.

The pressure sensor 132 outputs pressure data that can be stored in the memory device 114 and/or analyzed by the processor 112 of the control system 110. In some implementations, the pressure sensor 132 is an air pressure sensor (e.g., barometric pressure sensor) that generates sensor data indicative of the respiration (e.g., inhaling and/or exhaling) of the user 210 of the respiratory therapy system 120 and/or ambient pressure. In such implementations, the pressure sensor 132 can be coupled to or integrated in the respiratory therapy device 122. The pressure sensor 132 can be, for example, a capacitive sensor, an electromagnetic sensor, a piezoelectric sensor, a strain-gauge sensor, an optical sensor, a potentiometric sensor, or any combination thereof.

The flow rate sensor 134 outputs flow rate data that can be stored in the memory device 114 and/or analyzed by the processor 112 of the control system 110. In some implementations, the flow rate sensor 134 is used to determine an air flow rate from the respiratory therapy device 122, an air flow rate through the conduit 126, an air flow rate through the user interface 124, or any combination thereof. In such implementations, the flow rate sensor 134 can be coupled to or integrated in the respiratory therapy device 122, the user interface 124, or the conduit 126. The flow rate sensor 134 can be a mass flow rate sensor such as, for example, a rotary flow meter (e.g., Hall effect flow meters), a turbine flow meter, an orifice flow meter, an ultrasonic flow meter, a hot wire sensor, a vortex sensor, a membrane sensor, or any combination thereof.

The temperature sensor 136 outputs temperature data that can be stored in the memory device 114 and/or analyzed by the processor 112 of the control system 110. In some implementations, the temperature sensor 136 generates temperatures data indicative of a core body temperature of the user 210 (FIG. 2), a skin temperature of the user 210, a temperature of the air flowing from the respiratory therapy device 122 and/or through the conduit 126, a temperature in the user interface 124, an ambient temperature, or any combination thereof. The temperature sensor 136 can be, for example, a thermocouple sensor, a thermistor sensor, a silicon band gap temperature sensor or semiconductor-based sensor, a resistance temperature detector, or any combination thereof.

In some implementations, the respiratory therapy system 120 includes one or more microphones 140 communicatively coupled to the respiratory therapy system 120. Additionally, or alternatively, one or more microphones 140 may be part of the same larger system as the respiratory therapy system 120. In some implementations, the one or more microphones may include the microphone 140 as part of the acoustic sensor 141.

The one or more microphones 140 can be located at any location relative to the respiratory therapy system 120 and in acoustic communication with the airflow in the respiratory therapy system 120. For example, the respiratory therapy system 120 may include a microphone 140 (i) coupled externally to the conduit 126, (ii) positioned at least partially within the respiratory therapy device 122, (iii) coupled externally to the user interface 124, (iv) coupled directly or indirectly to a headgear associated with the user interface 124, or in any other suitable location. In some implementations, the one or more microphones 140 are coupled to a mobile device (for example, the user device 170 or a smart speaker(s) such as Google Home, Amazon Echo, Alexa etc.) that is communicatively coupled to the respiratory therapy system 120. In another implementation as shown in FIG. 3A, a microphone 340 is positioned on or at least partially outside of a housing 127 of the respiratory therapy device 122. In the implementation shown in FIG. 3A, the microphone 340 is at least partially movable relative to the housing 127 to aid in being directed to the user 210. For example, the microphone 340 can be rotated between about 5° and about 355° towards the user 210. In yet another implementation as shown in FIG. 3B, three microphones are coupled along the path of airflow in the respiratory therapy system 120—a first microphone 342 located between the blower motor 345 and the humidification tank 129, a second microphone 344 between the humidification tank 129 and the housing 127 of the respiratory therapy device 122, and a third microphone 346 adjacent to the junction between the user interface 124 and the conduit 126. As a general matter, presence of two or more microphones enhances leak detection by providing an enhanced estimation of the location of the leak, as further described below.

In some implementations, the one or more microphones 140 are configured to be in direct fluid communication with the airflow in the respiratory therapy system 120. For example, the one or more microphones 140 may be (i) positioned at least partially within the conduit 126, (ii) positioned at least partially within a component of the respiratory therapy device 122, which is in fluid communication with the conduit 126, (iii) positioned at least partially within the user interface 124, the user interface 124 being in fluid communication with the conduit 126, or (iv) any combination thereof. Further, in some implementations, the one or more microphones 140 are electrically connected with a circuit board (for example, connected physically, like mounted to a circuit board directly or indirectly) of the respiratory therapy device 122, which may be in acoustic communication (for example, via a small duct and/or a silicone window as in a stethoscope) or in fluid communication with the airflow in the respiratory therapy system 120.

The microphone 140 generates acoustic data that can be stored in the memory device 114 and/or analyzed by the processor 112 of the control system 110. The acoustic data generated by the microphone 140 is reproducible as one or more sound(s) during a sleep session (e.g., sounds from the user 210). The acoustic data from the microphone 140 can also be used to identify (e.g., using the control system 110) an event experienced by the user 210 during the sleep session.

The speaker 142 outputs sound waves that are audible to the user 210 of the system 100. The speaker 142 can be used, for example, as an alarm clock or to play an alert or message to the user 210 (e.g., in response to an event). In some implementations, the speaker 142 can be used to communicate the acoustic data generated by the microphone 140 to the user 210. The speaker 142 can be coupled to or integrated in the respiratory therapy device 122, the user interface 124, the conduit 126, or the user device 170.

The microphone 140 and the speaker 142 can be used as separate devices. In some implementations, the microphone 140 and the speaker 142 can be combined into an acoustic sensor 141 (which can be known as a sonar sensor), as described in, for example, WO 2018/050913 and WO 2020/104465, each of which is hereby incorporated by reference herein in its entirety. In such implementations, the speaker 142 generates or emits sound waves at a predetermined interval and/or frequency, and the microphone 140 detects the reflections of the emitted sound waves from the speaker 142. The sound waves generated or emitted by the speaker 142 have a frequency that is not audible to the human ear (e.g., below 20 Hz or above around 18 kHz) so as not to disturb the sleep of the user 210 or the bed partner 220 (FIG. 2). Based at least in part on the data from the microphone 140 and/or the speaker 142, the control system 110 can determine a location of the user 210 (FIG. 2) and/or one or more of the sleep-related parameters described in herein, such as, for example, a respiration signal, a respiration rate, an inspiration amplitude, an expiration amplitude, an inspiration-expiration ratio, a number of events per hour, a pattern of events, a sleep state, pressure settings of the respiratory therapy device 122, or any combination thereof. In this context, a sonar sensor may be understood to concern an active acoustic sensing, such as by generating/transmitting ultrasound or low frequency ultrasound sensing signals (e.g., in a frequency range of about 17-23 kHz, 18-22 kHz, or 17-18 kHz, for example), through the air. Such a system may be considered in relation to WO2018/050913 and WO 2020/104465 mentioned above. In some implementations, the speaker 142 is a bone conduction speaker. In some implementations, the one or more sensors 130 include (i) a first microphone that is the same or similar to the microphone 140, and is integrated into the acoustic sensor 141 and (ii) a second microphone that is the same as or similar to the microphone 140, but is separate and distinct from the first microphone that is integrated into the acoustic sensor 141.

The RF transmitter 148 generates and/or emits radio waves having a predetermined frequency and/or a predetermined amplitude (e.g., within a high frequency band, within a low frequency band, long wave signals, short wave signals, etc.). The RF receiver 146 detects the reflections of the radio waves emitted from the RF transmitter 148, and this data can be analyzed by the control system 110 to determine a location of the user 210 (FIG. 2) and/or one or more of the sleep-related parameters described herein. An RF receiver (either the RF receiver 146 and the RF transmitter 148 or another RF pair) can also be used for wireless communication between the control system 110, the respiratory therapy device 122, the one or more sensors 130, the user device 170, or any combination thereof. While the RF receiver 146 and RF transmitter 148 are shown as being separate and distinct elements in FIG. 1, in some implementations, the RF receiver 146 and RF transmitter 148 are combined as a part of an RF sensor 147 (which can be known as a radar sensor). In some such implementations, the RF sensor 147 includes a control circuit. The specific format of the RF communication can be WiFi, Bluetooth, or the like.

In some implementations, the RF sensor 147 is a part of a mesh system. One example of a mesh system is a WiFi mesh system, which can include mesh nodes, mesh router(s), and mesh gateway(s), each of which can be mobile/movable or fixed. In such implementations, the WiFi mesh system includes a WiFi router and/or a WiFi controller and one or more satellites (e.g., access points), each of which include an RF sensor that the is the same as, or similar to, the RF sensor 147. The WiFi router and satellites continuously communicate with one another using WiFi signals. The WiFi mesh system can be used to generate motion data based on changes in the WiFi signals (e.g., differences in received signal strength) between the router and the satellite(s) due to an object or person moving partially obstructing the signals. The motion data can be indicative of motion, breathing, heart rate, gait, falls, behavior, etc., or any combination thereof.

The camera 150 outputs image data reproducible as one or more images (e.g., still images, video images, thermal images, or a combination thereof) that can be stored in the memory device 114. The image data from the camera 150 can be used by the control system 110 to determine one or more of the sleep-related parameters described herein. For example, the image data from the camera 150 can be used to identify a location of the user 210, to determine a time when the user 210 enters the bed 230 (FIG. 2), and to determine a time when the user 210 exits the bed 230. In some implementations, the camera 150 includes a wide angle lens or a fish eye lens.

The infrared (IR) sensor 152 outputs infrared image data reproducible as one or more infrared images (e.g., still images, video images, or both) that can be stored in the memory device 114. The infrared data from the IR sensor 152 can be used to determine one or more sleep-related parameters during a sleep session, including a temperature of the user 210 and/or movement of the user 210. The IR sensor 152 can also be used in conjunction with the camera 150 when measuring the presence, location, and/or movement of the user 210. The IR sensor 152 can detect infrared light having a wavelength between about 700 nm and about 1 mm, for example, while the camera 150 can detect visible light having a wavelength between about 380 nm and about 740 nm.

The PPG sensor 154 outputs physiological data associated with the user 210 (FIG. 2) that can be used to determine one or more sleep-related parameters, such as, for example, a heart rate, a heart rate variability, a cardiac cycle, respiration rate, an inspiration amplitude, an expiration amplitude, an inspiration-expiration ratio, estimated blood pressure parameter(s), or any combination thereof. The PPG sensor 154 can be worn by the user 210, embedded in clothing and/or fabric that is worn by the user 210, embedded in and/or coupled to the user interface 124 and/or its associated headgear (e.g., straps, etc.), etc.

The ECG sensor 156 outputs physiological data associated with electrical activity of the heart of the user 210. In some implementations, the ECG sensor 156 includes one or more electrodes that are positioned on or around a portion of the user 210 during the sleep session. The physiological data from the ECG sensor 156 can be used, for example, to determine one or more of the sleep-related parameters described herein.

The EEG sensor 158 outputs physiological data associated with electrical activity of the brain of the user 210. In some implementations, the EEG sensor 158 includes one or more electrodes that are positioned on or around the scalp of the user 210 during the sleep session. The physiological data from the EEG sensor 158 can be used, for example, to determine a sleep state of the user 210 at any given time during the sleep session. In some implementations, the EEG sensor 158 can be integrated in the user interface 124 and/or the associated headgear (e.g., straps, etc.).

The capacitive sensor 160, the force sensor 162, and the strain gauge sensor 164 output data that can be stored in the memory device 114 and used by the control system 110 to determine one or more of the sleep-related parameters described herein. The EMG sensor 166 outputs physiological data associated with electrical activity produced by one or more muscles. The oxygen sensor 168 outputs oxygen data indicative of an oxygen concentration of gas (e.g., in the conduit 126 or at the user interface 124). The oxygen sensor 168 can be, for example, an ultrasonic oxygen sensor, an electrical oxygen sensor, a chemical oxygen sensor, an optical oxygen sensor, a pulse oximeter (e.g., SpO₂ sensor), or any combination thereof. In some implementations, the one or more sensors 130 also include a galvanic skin response (GSR) sensor, a blood flow sensor, a respiration sensor, a pulse sensor, a sphygmomanometer sensor, an oximetry sensor, or any combination thereof.

The analyte sensor 174 can be used to detect the presence of an analyte in the exhaled breath of the user 210. The data output by the analyte sensor 174 can be stored in the memory device 114 and used by the control system 110 to determine the identity and concentration of any analytes in the breath of the user 210. In some implementations, the analyte sensor 174 is positioned near a mouth of the user 210 to detect analytes in breath exhaled from the user 210's mouth. For example, when the user interface 124 is a facial mask that covers the nose and mouth of the user 210, the analyte sensor 174 can be positioned within the facial mask to monitor the user 210's mouth breathing. In other implementations, such as when the user interface 124 is a nasal mask or a nasal pillow mask, the analyte sensor 174 can be positioned near the nose of the user 210 to detect analytes in breath exhaled through the user's nose. In still other implementations, the analyte sensor 174 can be positioned near the user 210's mouth when the user interface 124 is a nasal mask or a nasal pillow mask. In this implementation, the analyte sensor 174 can be used to detect whether any air is inadvertently leaking from the user 210's mouth. In some implementations, the analyte sensor 174 is a volatile organic compound (VOC) sensor that can be used to detect carbon-based chemicals or compounds. In some implementations, the analyte sensor 174 can also be used to detect whether the user 210 is breathing through their nose or mouth. For example, if the data output by an analyte sensor 174 positioned near the mouth of the user 210 or within the facial mask (in implementations where the user interface 124 is a facial mask) detects the presence of an analyte, the control system 110 can use this data as an indication that the user 210 is breathing through their mouth.

The moisture sensor 176 outputs data that can be stored in the memory device 114 and used by the control system 110. The moisture sensor 176 can be used to detect moisture in various areas surrounding the user (e.g., inside the conduit 126 or the user interface 124, near the user 210's face, near the connection between the conduit 126 and the user interface 124, near the connection between the conduit 126 and the respiratory therapy device 122, etc.). Thus, in some implementations, the moisture sensor 176 can be coupled to or integrated in the user interface 124 or in the conduit 126 to monitor the humidity of the pressurized air from the respiratory therapy device 122. In other implementations, the moisture sensor 176 is placed near any area where moisture levels need to be monitored. The moisture sensor 176 can also be used to monitor the humidity of the ambient environment surrounding the user 210, for example, the air inside the bedroom.

The Light Detection and Ranging (LiDAR) sensor 178 can be used for depth sensing. This type of optical sensor (e.g., laser sensor) can be used to detect objects and build three dimensional (3D) maps of the surroundings, such as of a living space. LiDAR can generally utilize a pulsed laser to make time of flight measurements. LiDAR is also referred to as 3D laser scanning. In an example of use of such a sensor, a fixed or mobile device (such as a smartphone) having a LiDAR sensor 178 can measure and map an area extending 5 meters or more away from the sensor. The LiDAR data can be fused with point cloud data estimated by an electromagnetic RADAR sensor, for example. The LiDAR sensor(s) 178 can also use artificial intelligence (AI) to automatically geofence RADAR systems by detecting and classifying features in a space that might cause issues for RADAR systems, such a glass windows (which can be highly reflective to RADAR). LiDAR can also be used to provide an estimate of the height of a person, as well as changes in height when the person sits down, or falls down, for example. LiDAR may be used to form a 3D mesh representation of an environment. In a further use, for solid surfaces through which radio waves pass (e.g., radio-translucent materials), the LiDAR may reflect off such surfaces, thus allowing a classification of different type of obstacles.

While shown separately in FIG. 1, any combination of the one or more sensors 130 can be integrated in and/or coupled to any one or more of the components of the system 100, including the respiratory therapy device 122, the user interface 124, the conduit 126, the humidification tank 129, the control system 110, the user device 170, or any combination thereof. For example, the acoustic sensor 141 and/or the RF sensor 147 can be integrated in and/or coupled to the user device 170. In such implementations, the user device 170 can be considered a secondary device that generates additional or secondary data for use by the system 100 (e.g., the control system 110) according to some aspects of the present disclosure. In some implementations, the pressure sensor 132 and/or the flow rate sensor 134 are integrated into and/or coupled to the respiratory therapy device 122. In some implementations, at least one of the one or more sensors 130 is not coupled to the respiratory therapy device 122, the control system 110, or the user device 170, and is positioned generally adjacent to the user 210 during the sleep session (e.g., positioned on or in contact with a portion of the user 210, worn by the user 210, coupled to or positioned on the nightstand 240, coupled to the mattress 232, coupled to the ceiling, etc.). More generally, the one or more sensors 130 can be positioned at any suitable location relative to the user 210 such that the one or more sensors 130 can generate physiological data associated with the user 210 and/or the bed partner 220 during one or more sleep session.

The data from the one or more sensors 130 can be analyzed to determine one or more sleep-related parameters, which can include a respiration signal, a respiration rate, a respiration pattern, an inspiration amplitude, an expiration amplitude, an inspiration-expiration ratio, an occurrence of one or more events, a number of events per hour, a pattern of events, an average duration of events, a range of event durations, a ratio between the number of different events, a sleep stage, an apnea-hypopnea index (AHI), or any combination thereof. The one or more events can include snoring, apneas, central apneas, obstructive apneas, mixed apneas, hypopneas, an intentional user interface leak, an unintentional user interface leak, a mouth leak, a cough, a restless leg, a sleeping disorder, choking, an increased heart rate, labored breathing, an asthma attack, an epileptic episode, a seizure, increased blood pressure, or any combination thereof. Many of these sleep-related parameters are physiological parameters, although some of the sleep-related parameters can be considered to be non-physiological parameters. Other types of physiological and non-physiological parameters can also be determined, either from the data from the one or more sensors 130, or from other types of data.

The user device 170 (FIG. 1) includes a display device 172. The user device 170 can be, for example, a mobile device such as a smart phone, a tablet, a laptop, or the like. Alternatively, the user device 170 can be an external sensing system, a television (e.g., a smart television) or another smart home device (e.g., a smart speaker(s) such as Google Home, Amazon Echo, Alexa etc.). In some implementations, the user device 170 is a wearable device (e.g., a smart watch). The display device 172 is generally used to display image(s) including still images, video images, or both. In some implementations, the display device 172 acts as a human-machine interface (HMI) that includes a graphic user interface (GUI) configured to display the image(s) and an input interface. The display device 172 can be an LED display, an OLED display, an LCD display, or the like. The input interface can be, for example, a touchscreen or touch-sensitive substrate, a mouse, a keyboard, or any sensor system configured to sense inputs made by a human user interacting with the user device 170. In some implementations, one or more user devices 170 can be used by and/or included in the system 100.

The blood pressure device 180 is generally used to aid in generating physiological data for determining one or more blood pressure measurements associated with the user 210. The blood pressure device 180 can include at least one of the one or more sensors 130 to measure, for example, a systolic blood pressure component and/or a diastolic blood pressure component.

In some implementations, the blood pressure device 180 is a sphygmomanometer including an inflatable cuff that can be worn by the user 210 and a pressure sensor (e.g., the pressure sensor 132 described herein). For example, as shown in the example of FIG. 2, the blood pressure device 180 can be worn on an upper arm of the user 210. In such implementations where the blood pressure device 180 is a sphygmomanometer, the blood pressure device 180 also includes a pump (e.g., a manually operated bulb) for inflating the cuff. In some implementations, the blood pressure device 180 is coupled to the respiratory therapy device 122 of the respiratory therapy system 120, which in turn delivers pressurized air to inflate the cuff. More generally, the blood pressure device 180 can be communicatively coupled with, and/or physically integrated in (e.g., within a housing), the control system 110, the memory device 114, the respiratory therapy system 120, the user device 170, and/or the activity tracker 190.

The activity tracker 190 is generally used to aid in generating physiological data for determining an activity measurement associated with the user 210. The activity measurement can include, for example, a number of steps, a distance traveled, a number of steps climbed, a duration of physical activity, a type of physical activity, an intensity of physical activity, time spent standing, a respiration rate, an average respiration rate, a resting respiration rate, a maximum respiration rate, a respiration rate variability, a heart rate, an average heart rate, a resting heart rate, a maximum heart rate, a heart rate variability, a number of calories burned, blood oxygen saturation, electrodermal activity (also known as skin conductance or galvanic skin response), or any combination thereof. The activity tracker 190 includes one or more of the sensors 130 described herein, such as, for example, the motion sensor 138 (e.g., one or more accelerometers and/or gyroscopes), the PPG sensor 154, and/or the ECG sensor 156.

In some implementations, the activity tracker 190 is a wearable device that can be worn by the user 210, such as a smartwatch, a wristband, a ring, or a patch. For example, referring to FIG. 2, the activity tracker 190 is worn on a wrist of the user 210. The activity tracker 190 can also be coupled to or integrated a garment or clothing that is worn by the user 210. Alternatively, still, the activity tracker 190 can also be coupled to or integrated in (e.g., within the same housing) the user device 170. More generally, the activity tracker 190 can be communicatively coupled with, or physically integrated in (e.g., within a housing), the control system 110, the memory device 114, the respiratory therapy system 120, the user device 170, and/or the blood pressure device 180.

Referring back to FIG. 1, while the control system 110 and the memory device 114 are described and shown in FIG. 1 as being a separate and distinct component of the system 100, in some implementations, the control system 110 and/or the memory device 114 are integrated in the user device 170 and/or the respiratory therapy device 122. Alternatively, in some implementations, the control system 110 or a portion thereof (e.g., the processor 112) can be located in a cloud (e.g., integrated in a server, integrated in an Internet of Things (IoT) device, connected to the cloud, be subject to edge cloud processing, etc.), located in one or more servers (e.g., remote servers, local servers, etc., or any combination thereof.

While system 100 is shown as including all of the components described above, more or fewer components can be included in a system according to implementations of the present disclosure. For example, a first alternative system includes the control system 110, the memory device 114, and at least one of the one or more sensors 130 and does not include the respiratory therapy system 120. As another example, a second alternative system includes the control system 110, the memory device 114, at least one of the one or more sensors 130, and the user device 170. As yet another example, a third alternative system includes the control system 110, the memory device 114, the respiratory therapy system 120, at least one of the one or more sensors 130, and the user device 170. Thus, various systems can be formed using any portion or portions of the components shown and described herein and/or in combination with one or more other components.

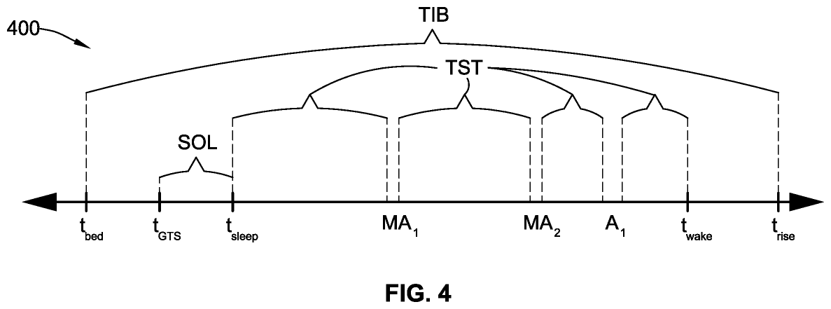

Referring to FIG. 4, an exemplary timeline 400 for a sleep session is illustrated. The timeline 400 includes an enter bed time (t_{bed}), a go-to-sleep time (t_{GTS}), an initial sleep time (tₛₗₑₑₚ), a first micro-awakening MA₁ and a second micro-awakening MA₂, a wake-up time (t_{wake}), and a rising time (tᵣᵢₛₑ). As used herein, a sleep session can be defined in multiple ways. For example, a sleep session can be defined by an initial start time and an end time. In some implementations, a sleep session is a duration where the user 210 is asleep, that is, the sleep session has a start time and an end time, and during the sleep session, the user 210 does not wake until the end time. That is, any period of the user 210 being awake is not included in a sleep session. From this first definition of sleep session, if the user 210 wakes ups and falls asleep multiple times in the same night, each of the sleep intervals separated by an awake interval is a sleep session.

Alternatively, in some implementations, a sleep session has a start time and an end time, and during the sleep session, the user 210 can wake up, without the sleep session ending, so long as a continuous duration that the user 210 is awake is below an awake duration threshold. The awake duration threshold can be defined as a percentage of a sleep session. The awake duration threshold can be, for example, about twenty percent of the sleep session, about fifteen percent of the sleep session duration, about ten percent of the sleep session duration, about five percent of the sleep session duration, about two percent of the sleep session duration, etc., or any other threshold percentage. In some implementations, the awake duration threshold is defined as a fixed amount of time, such as, for example, about one hour, about thirty minutes, about fifteen minutes, about ten minutes, about five minutes, about two minutes, etc., or any other amount of time.

In some implementations, a sleep session is defined as the entire time between the time in the evening at which the user 210 first entered the bed 230, and the time the next morning when user 210 last left the bed 230. Put another way, a sleep session can be defined as a period of time that begins on a first date (e.g., Monday, Jan. 6, 2020) at a first time (e.g., 10:00 PM), that can be referred to as the current evening, when the user 210 first enters the bed 230 with the intention of going to sleep (e.g., not if the user 210 intends to first watch television or play with a smart phone before going to sleep, etc.), and ends on a second date (e.g., Tuesday, Jan. 7, 2020) at a second time (e.g., 7:00 AM), that can be referred to as the next morning, when the user 210 first exits the bed 230 with the intention of not going back to sleep that next morning.

In some implementations, the user can manually define the beginning of a sleep session and/or manually terminate a sleep session. For example, the user 210 can select (e.g., by clicking or tapping) one or more user-selectable element that is displayed on the display device 172 of the user device 170 (FIG. 1) to manually initiate or terminate the sleep session.

Referring to the timeline 400 in FIG. 4, the enter bed time t_{bed} is associated with the time that the user 210 initially enters the bed 230 prior to falling asleep (e.g., when the user 210 lies down or sits in the bed 230). The enter bed time t_{bed} can be identified based on a bed threshold duration to distinguish between times when the user 210 enters the bed 230 for sleep and when the user 210 enters the bed 230 for other reasons (e.g., to watch TV). For example, the bed threshold duration can be at least about 10 minutes, at least about 20 minutes, at least about 30 minutes, at least about 45 minutes, at least about 1 hour, at least about 2 hours, etc. While the enter bed time t_{bed} is described herein in reference to a bed, more generally, the enter time t_{bed} can refer to the time the user 210 initially enters any location for sleeping (e.g., a couch, a chair, a sleeping bag, etc.).

The go-to-sleep time (GTS) is associated with the time that the user 210 initially attempts to fall asleep after entering the bed 230 (t_{bed}). For example, after entering the bed 230, the user 210 may engage in one or more activities to wind down prior to trying to sleep (e.g., reading, watching TV, listening to music, using the user device 170, etc.). The initial sleep time (tₛₗₑₑₚ) is the time that the user 210 initially falls asleep. For example, the initial sleep time (tₛₗₑₑₚ) can be the time that the user 210 initially enters the first non-REM sleep stage.

The wake-up time t_{wake} is the time associated with the time when the user 210 wakes up without going back to sleep (e.g., as opposed to the user waking up in the middle of the night and going back to sleep). The user 210 may experience one of more unconscious microawakenings (e.g., microawakenings MA₁ and MA₂) having a short duration (e.g., 5 seconds, 10 seconds, 30 seconds, 1 minute, etc.) after initially falling asleep. In contrast to the wake-up time t_{wake}, the user 210 goes back to sleep after each of the microawakenings MA₁ and MA₂. Similarly, the user 210 may have one or more conscious awakenings (e.g., awakening A) after initially falling asleep (e.g., getting up to go to the bathroom, attending to children or pets, sleep walking, etc.). However, the user 210 goes back to sleep after the awakening A. Thus, the wake-up time t_{wake} can be defined, for example, based on a wake threshold duration (e.g., the user 210 is awake for at least 15 minutes, at least 20 minutes, at least 30 minutes, at least 1 hour, etc.).

Similarly, the rising time tᵣᵢₛₑ is associated with the time when the user 210 exits the bed 230 and stays out of the bed 230 with the intent to end the sleep session (e.g., as opposed to the user 210 getting up during the night to go to the bathroom, to attend to children or pets, sleep walking, etc.). In other words, the rising time tᵣᵢₛₑ is the time when the user 210 last leaves the bed 230 without returning to the bed 230 until a next sleep session (e.g., the following evening). Thus, the rising time tᵣᵢₛₑ can be defined, for example, based on a rise threshold duration (e.g., the user 210 has left the bed 230 for at least 15 minutes, at least 20 minutes, at least 30 minutes, at least 1 hour, etc.). The enter bed time t_{bed} time for a second, subsequent sleep session can also be defined based on a rise threshold duration (e.g., the user has left the bed for at least 4 hours, at least 6 hours, at least 8 hours, at least 12 hours, etc.).

As described above, the user 210 may wake up and get out of bed 230 one more times during the night between the initial t_{bed} and the final tᵣᵢₛₑ. In some implementations, the final wake-up time t_{wake} and/or the final rising time tᵣᵢₛₑ that are identified or determined based on a predetermined threshold duration of time subsequent to an event (e.g., falling asleep or leaving the bed). Such a threshold duration can be customized for the user 210. For a standard user 210 who goes to bed in the evening, then wakes up and goes out of bed in the morning any period (between the user 210 waking up (t_{wake}) or raising up (tᵣᵢₛₑ), and the user 210 either going to bed (t_{bed}), going to sleep (t_{GTS}) or falling asleep (tₛₗₑₑₚ) of between about 12 and about 18 hours can be used. For users that spend longer periods of time in bed, shorter threshold periods may be used (e.g., between about 8 hours and about 14 hours). The threshold period may be initially selected and/or later adjusted based on the system monitoring the user's sleep behavior.

The total time in bed (TIB) is the duration of time between the time enter bed time t_{bed} and the rising time tᵣᵢₛₑ. The total sleep time (TST) is associated with the duration between the initial sleep time and the wake-up time, excluding any conscious or unconscious awakenings and/or micro-awakenings therebetween. Generally, the total sleep time (TST) will be shorter than the total time in bed (TIB) (e.g., one minute short, ten minutes shorter, one hour shorter, etc.). For example, referring to the timeline 400 of FIG. 4, the total sleep time (TST) spans between the initial sleep time tₛₗₑₑₚ and the wake-up time t_{wake}, but excludes the duration of the first micro-awakening MA₁, the second micro-awakening MA₂, and the awakening A. As shown, in this example, the total sleep time (TST) is shorter than the total time in bed (TIB).

In some implementations, the total sleep time (TST) can be defined as a persistent total sleep time (PTST). In such implementations, the persistent total sleep time excludes a predetermined initial portion or period of the first non-REM stage (e.g., light sleep stage). For example, the predetermined initial portion can be between about 30 seconds and about 20 minutes, between about 1 minute and about 10 minutes, between about 3 minutes and about 5 minutes, etc. The persistent total sleep time is a measure of sustained sleep, and smooths the sleep-wake hypnogram. For example, when the user 210 is initially falling asleep, the user 210 may be in the first non-REM stage for a very short time (e.g., about 30 seconds), then back into the wakefulness stage for a short period (e.g., one minute), and then goes back to the first non-REM stage. In this example, the persistent total sleep time excludes the first instance (e.g., about 30 seconds) of the first non-REM stage.

In some implementations, the sleep session is defined as starting at the enter bed time (t_{bed}) and ending at the rising time (tᵣᵢₛₑ), i.e., the sleep session is defined as the total time in bed (TIB). In some implementations, a sleep session is defined as starting at the initial sleep time (tₛₗₑₑₚ) and ending at the wake-up time (t_{wake}). In some implementations, the sleep session is defined as the total sleep time (TST). In some implementations, a sleep session is defined as starting at the go-to-sleep time (t_{GTS}) and ending at the wake-up time (t_{wake}). In some implementations, a sleep session is defined as starting at the go-to-sleep time (t_{GTS}) and ending at the rising time (tᵣᵢₛₑ). In some implementations, a sleep session is defined as starting at the enter bed time (t_{bed}) and ending at the wake-up time (t_{wake}). In some implementations, a sleep session is defined as starting at the initial sleep time (tₛₗₑₑₚ) and ending at the rising time (tᵣᵢₛₑ).

In some implementations, one or more of the sensors 130 can be used to determine or identify the enter bed time (t_{bed}), the go-to-sleep time (t_{GTS}), the initial sleep time (tₛₗₑₑₚ), one or more first micro-awakenings (e.g., MA₁ and MA₂), the wake-up time (t_{wake}), the rising time (tᵣᵢₛₑ), or any combination thereof, which in turn define the sleep session. For example, the enter bed time t_{bed} can be determined based on, for example, data generated by the motion sensor 138, the microphone 140, the camera 150, or any combination thereof. The go-to-sleep time can be determined based on, for example, data from the motion sensor 138 (e.g., data indicative of no movement by the user 210), data from the camera 150 (e.g., data indicative of no movement by the user 210 and/or that the user 210 has turned off the lights) data from the microphone 140 (e.g., data indicative of the user 210 turning off a TV), data from the user device 170 (e.g., data indicative of the user 210 no longer using the user device 170), data from the pressure sensor 132 and/or the flow rate sensor 134 (e.g., data indicative of the user 210 turning on the respiratory therapy device 122, data indicative of the user 210 donning the user interface 124, etc.), or any combination thereof.

The systems and methods described herein can be configured to detect one or more leaks in the respiratory therapy system 120 using the acoustic data. In some implementations, the leak is an unintentional leak that is indicative of (i) airflow leaking between the user 210 and the user interface 124, (ii) airflow leaking from a mouth of the user 210, (iii) airflow leaking between the user interface 124 and the conduit 126, (iv) airflow leaking between the conduit 126 and the respiratory therapy device 122, or (v) any combination thereof. The other implementations, the leak is an intentional leak that is indicative of airflow venting from the one or more vents 125 positioned in the user interface 124, the one or more vents in the humidification tank 129, or one or more vents in a waterless humidification filter on the conduit 126 of the respiratory therapy system 120.

Occurrence of the leak emits an acoustic signal in the form of a sound that is detected by the one or more microphones 140 (such as the microphone 340 shown in FIG. 3A, the microphones 342, 344, 346 in FIG. 3B) to generate the acoustic data. Thus, as used herein, the “noise” associated with operation of the respiratory therapy system 120 can include audible noise due to the occurrence of the leak, inaudible noise due to the occurrence of the leak such as an inaudible frequency (e.g., the frequency being outside of the frequency range for human hearing) and/or an inaudible amplitude (e.g., the amplitude being low enough that the sound is not loud enough for human perception), an audible acoustic signal propagating in the conduit 126 or in other components of the respiratory therapy device 122, an inaudible acoustic signal propagating in the conduit 126 or in other components of the respiratory therapy device 122, or any combination thereof.

In some implementations, the acoustic signal is detected at specific times, such as before the user 210 first puts on the user interface 124, when the user 210 first puts on the user interface 124, during a particular phase of a breathing cycle of the user (for example, during an expiration phase of breathing when airflow leak from the mouth is common) detected based on flow data from the flow rate sensor 134 and pressure data from the pressure sensor 132, and/or at predetermined time intervals. In some implementations, the specific monitoring times may be selected to be at intervals of about 0.1-2 seconds for a duration of about 0.02-4 seconds. Such discontinuous (e.g., intermittent) sampling ensures that the content of any speech of the user 210 or the bed partner 220 present in the collected acoustic data remains unintelligible and private. Privacy can also be ensured by not retaining the acoustic data once the leak determination is made or, by processing the acoustic data locally without outsourcing it away from the respiratory therapy device 122 or the user device 170.

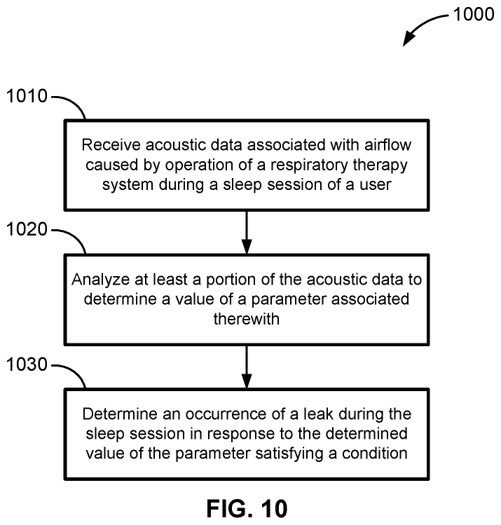

A method 1000 of detecting the leak in the respiratory therapy system 120 is described below and illustrated with respect to a flow diagram shown in FIG. 10. Referring to the flow diagram of FIG. 10, at step 1010 of method 1000, acoustic data associated with airflow caused by operation of the respiratory therapy system 120 during a sleep session (alternatively during setup of the respiratory therapy system 120) of the user 210 is received. The acoustic data is generated by the one or more microphones 140 (such as the microphone 340 shown in FIG. 3A, the microphones 342, 344, 346 in FIG. 3B), as discussed above.

The acoustic data generated by the one or more microphones 140 is representative of the acoustic signal received from the ambient environment, airflow through the respiratory therapy system 120 and presence or absence of a leak in the airflow. For example, if any type of leak is occurring in the respiratory therapy system 120 leading to a corresponding noise being produced, such a noise can be detected by the one or more microphones 140, and represented by the generated acoustic data. In general, in order to detect the leak in the respiratory therapy system 120, the noise represented by the acoustic data is noise caused by the occurrence of the leak somewhere in the respiratory therapy system 120, such as, airflow leaking between the user 210 and the user interface 124, from a mouth of the user 210, between the user interface 124 and the conduit 126, between the conduit 126 and the respiratory therapy device 122, etc.

Under normal operating conditions without occurrence of a leak, acoustic data collected at each of the one or more microphones 140 will exhibit unique baseline characteristics that depend on factors such as, but not limited to, (i) the geometry of the path of airflow in the respiratory therapy system 120, (ii) the components forming the path of airflow (defined by the respiratory therapy device 122, the conduit 126, and the user interface 124), (iii) the speed of the blower motor 345 in the respiratory therapy device 122 (FIG. 3B), (iv) the design of the humidification tank 129 or another humidifier (e.g., size, materials, water level, etc.) along the path of airflow, or (v) any combination thereof. These baseline characteristics may be pre-determined (e.g., estimated or measured in the factory) or learned in use over time during known periods of no leak.

As an example, referring again to the non-limiting implementation of FIG. 3B, each of the different microphones 342, 344, 346 would correspondingly show unique baseline characteristics. The baseline characteristic of the microphone 342 would mostly include acoustic data from the blower motor 345 (which typically generates frequencies from 0 to 20 kHz at least) in the respiratory therapy device 122, and thus yield an approximately pink and/or brown spectrum with relatively large amplitude. The baseline characteristic of the microphone 344 would have a spectrum similar to the baseline characteristic of the microphone 342, but any high frequencies may be removed by the humidification tank 129 or another humidifier acting as a low-pass filter, and reduce the relatively large amplitude of the acoustic data received. The high frequencies thus removed depends on the design of the humidification tank 129 or the another humidifier, but typically would have a cut-off point (e.g., a 3 dB reduction) in the range of about 0.5-5 kHz, and any frequencies above that are gradually more supressed. The baseline characteristic of the microphone 346 would have a spectrum similar to the microphone 344, but with the addition of acoustic data due to intentional flow at the one or more vents 125 of the user interface 124.

At step 1020 of method 1000, at least a portion of the acoustic data is analyzed to determine a value of a parameter associated with airflow in the respiratory therapy system 120. In some implementations, the parameter associated with airflow in the respiratory therapy system 120 may be acoustic intensity, acoustic volume, acoustic frequency, acoustic energy ratio, or any combination thereof.

A variety of different techniques can be used to analyze the acoustic data. In some implementations, the noise caused by the occurrence of the leak results in a specific acoustic pattern or acoustic signature in the acoustic data. Generally, an acoustic signature can be any feature or combination of features in the acoustic data that are caused by a certain type of noise. The acoustic signature is generally unique to the certain type of noise and may include a maximum value, a rate of change, a standard deviation, a range, or any combination thereof associated with the parameter in the acoustic data such as acoustic intensity, acoustic volume, acoustic frequency, acoustic energy ratio, or any combination thereof. In some implementations, one or more features of the frequency spectrum that are indicative of the occurrence of the leak may define the acoustic signature. An acoustic pattern could be any type of periodic (e.g., repeating) feature or features in the acoustic data that results from a certain types of noise. In some implementations, the acoustic pattern resulting from a certain type of noise is the acoustic signature of that noise. In other implementations, the acoustic pattern resulting from a certain type of noise may be shared between multiple types of noise. The acoustic data can be analyzed to identify this acoustic pattern or acoustic signature of the noise caused by the occurrence of the leak.

One technique for analyzing the acoustic data includes generating time-domain measurements, such as a measurement representing the intensity of the detected noise versus time. The intensity of the noise can be analyzed to detect an acoustic pattern or acoustic signature indicative of noise caused by the occurrence of the leak. In one example, the leak may cause a distinct noise, which is represented by a periodic intensity pattern. This periodic intensity pattern can be identified to detect the occurrence of the leak.

Additionally, or alternatively, a volume level of the noise caused by the occurrence of the leak can be determined from a measurement representing the intensity of the noise versus time. If the volume reaches a sufficient threshold volume, it can be determined that there is a leak. In some implementations, the volume of the noise is compared to a baseline volume level. The baseline volume level can be determined by measuring the noise of the respiratory therapy system 120 when it is known that there is no leak. If the volume level of the detected noise exceeds the baseline volume level by a sufficient amount, it can be determined that there is a leak from the respiratory therapy system 120. Such a leak may be an unintentional leak that removes an undesired amount of pressurized air from the airflow in the respiratory therapy system 120.

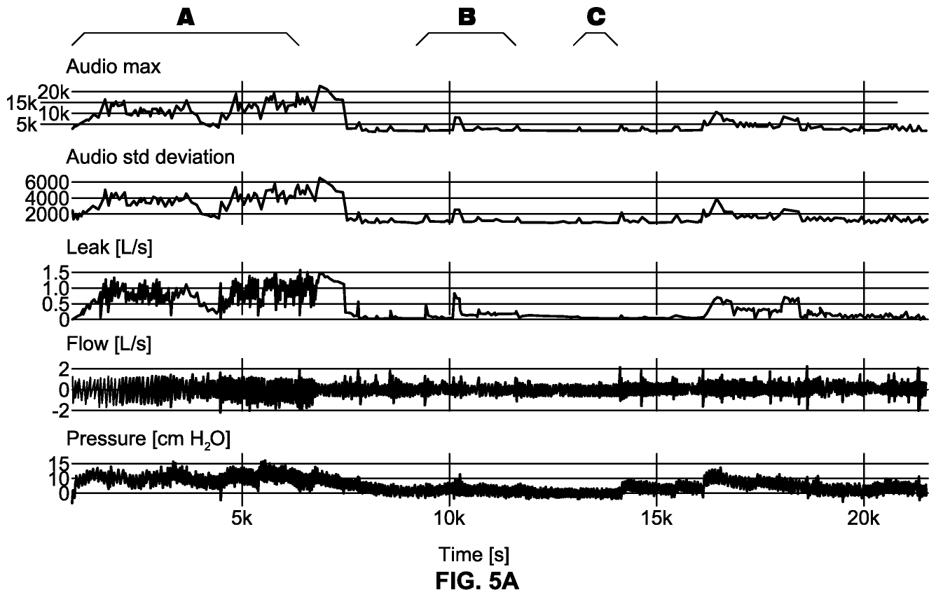

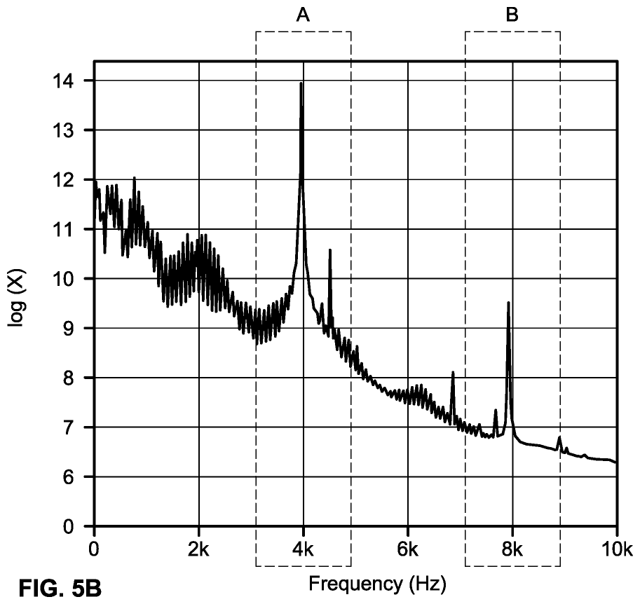

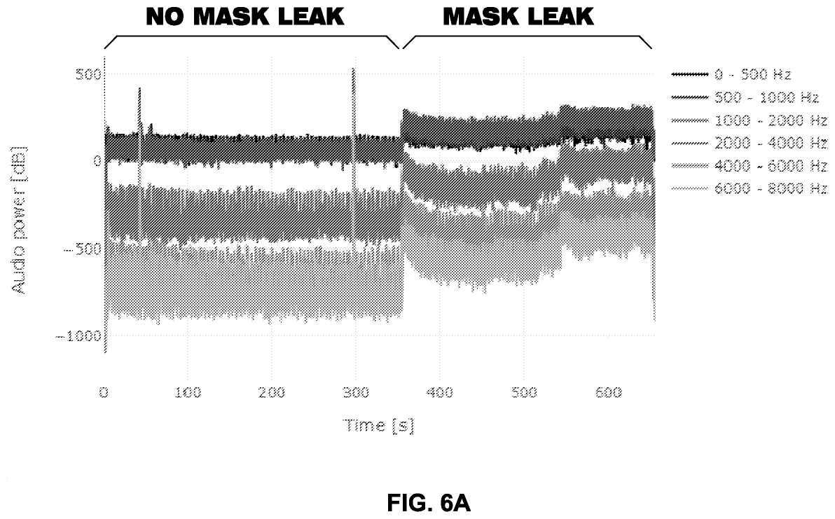

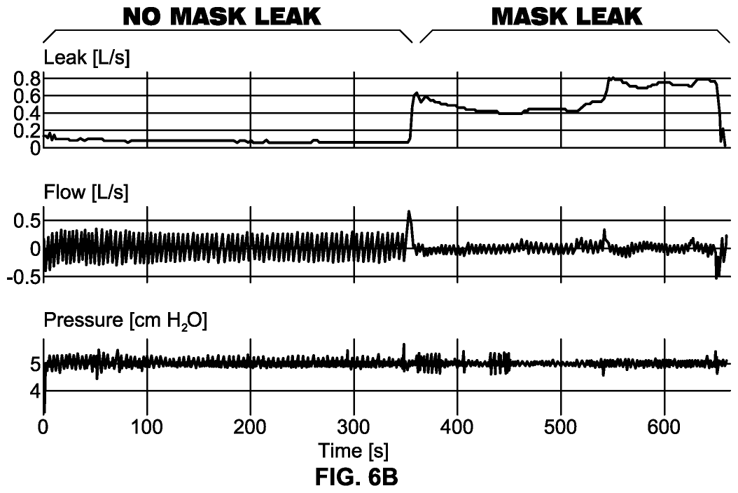

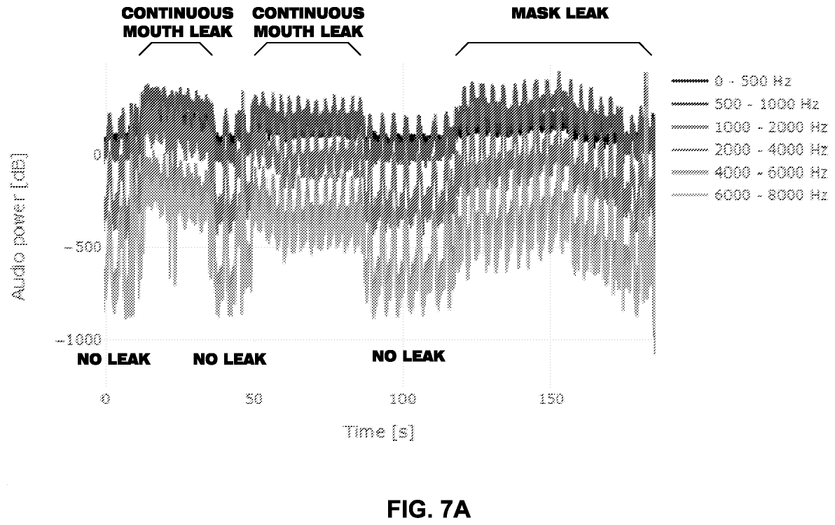

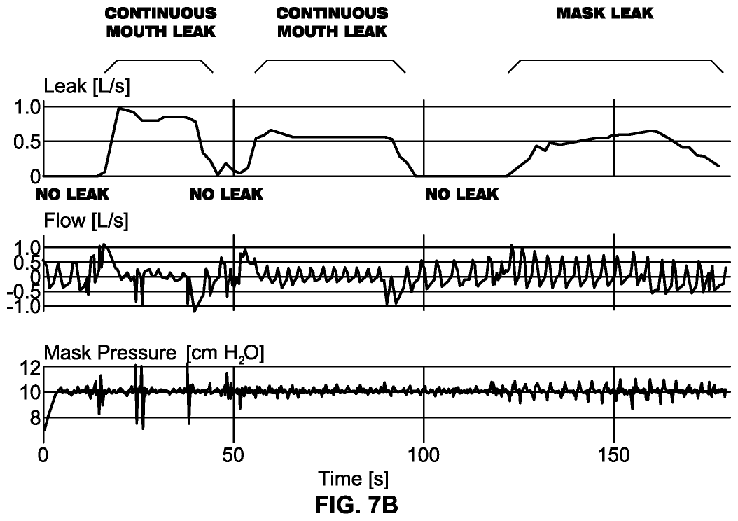

FIG. 5A illustrates a comparative graphical representation of pressure in the user interface 124 (measured in cm H₂O), flow rate (measured in liters per second), leak rate (measured in liters per second), standard deviation of acoustic intensity, and maximum value of acoustic intensity over a time period of more than 20,000 seconds during which leaks occur in the respiratory therapy system 100. Acoustic intensity is one of the parameters determined from the acoustic data in FIG. 5A generated by the one or more microphones 140 positioned with the respiratory therapy device 122. Statistical data associated with the parameter such as, but not limited to, standard deviation of acoustic intensity, maximum value of acoustic intensity, and percentiles of acoustic intensity are extracted from the acoustic data sampled over predetermined intervals (for example, one second) throughout overlapping or non-overlapping windows of time within the time period. The statistical data collected over the time period can then be low-pass filtered (for example, by a rolling average or applying a digital filter such as an finite impulse response (FIR) or an infinite impulse response (IIR)). Occurrence of a leak is determined based on whether the parameter satisfies a condition (for example, being above a predetermined threshold) as described below. As shown in FIG. 5A, the statistical data can be plotted with the pressure in the user interface 124, flow rate, and leak rate over the time period. The comparative graphical representation in FIG. 5A shows a clear correlation among the statistical data for acoustic intensity, flow rate, pressure in the user interface 124, and the leak rate. The correlation is commensurate with typical errors associated with inaccurate estimation of impedance of airflow within the respiratory therapy system 120. The graphical representation of FIG. 5A illustrates a first segment showing no leak (inset C) as well as a second segment showing high levels of leak (inset A) and a third segment showing relatively low levels of leak (inset B). In different implementations, another parameter such as acoustic energy ratios in different frequency bands, may be used to extract statistical data from acoustic data generated by the microphone 140, as described with respect to FIGS. 6A-6B and FIGS. 7A-7B.

Another technique for analyzing the acoustic data includes generating frequency-domain measurements, such as a frequency spectrum that represents the intensity of the detected noise versus frequency. In some implementations, the frequency spectrum can be obtained by taking the Fourier transform of the measurement representing the intensity of the detected noise versus time. The frequency spectrum can be analyzed to identify various features representing the noise caused by the occurrence of the leak. For example, the resulting noise due to the leak may have a distinct frequency, or be composed of multiple frequencies within distinct frequency range. This frequency or frequency range can be identified from the frequency spectrum in order to determine that there is leak in the airflow within the respiratory therapy system 120. Such a leak may be an unintentional leak as indicated by acoustic features that can be derived by taking, for example, acoustic energy ratios in the different frequency ranges.

A further technique for analyzing the acoustic data includes utilizing cepstrum analysis. A cepstrum can be considered as a spectrum of a spectrum, and can be obtained in some implementations by taking the inverse Fourier Transform of the logarithm of the frequency spectrum. In some implementations, the frequency spectrum is plotted on the mel scale. The mel scale is a warped version of a linear frequency scale, where the difference between consecutive frequency intervals is not equally-spaced as the frequency increases. The mel scale generally approximates the response of the human auditory scale more accurately than a linear frequency scale. When utilizing a frequency spectrum plotted on the mel scale, a discrete cosine transform of the frequency spectrum can be taken (instead of the inverse Fourier Transform), to obtain a mel-frequency cepstrum. After the mel-frequency cepstrum has been generated, the mel-frequency cepstral coefficients can be determined from the mel-frequency cepstrum. The mel-frequency cepstral coefficients are the amplitudes of the components of the spectrum.

The mel-frequency cepstral coefficients can be correlated with different noises in order to detect when different types of leaks (such as unintentional leak through the mouth of the user 210 or the user interface 124) in the airflow in the respiratory therapy system 120 is causing noise. In some implementations, a machine learning model (such as a convolutional neural network) can be trained to detect noise associated with the different types of the leak. The machine learning model may be trained on data from respiratory therapy systems with known location and/or source of leaks, levels of leak, etc. The training data may be collected for a comprehensive range of therapy settings having different pressures, flow rates, etc., and using different respiratory devices, conduits, user interfaces, etc.

In such implementations, data representing mel-frequency cepstral coefficients known to result from the operation of the respiratory therapy system 120 when a leak is present is inputted into the machine learning model. Once the machine learning model has been sufficiently trained, the machine learning model can accurately determine whether new mel-frequency cepstral coefficients represent noise caused by each of the different types of the leak (such as unintentional leak through the mouth of the user 210 or the user interface 124) in the airflow within the respiratory therapy system 120.

Referring back to the method 1000, at step 1030, the occurrence of the leak during the sleep session is determined in response to the determined value of the parameter satisfying a condition. Determination of the occurrence of the leak may include determining the presence of the leak, determining a type of leak (for example, whether the leak is unintentional or intentional leak as recognized from the acoustic data and subsequent analyses discussed above), an amount of the leak based at least partially on the acoustic data, a location of the leak in the respiratory therapy system 120, and the other information associated with the leak. In some implementations, satisfaction of the condition by the parameter may include exceeding a threshold value, not exceeding the threshold value of the parameter, staying within a predetermined range of values of the parameter, staying outside the predetermined range of values of the parameter, or any combination thereof.

In some implementations, if the leak is determined to be an unintentional leak, the amount of unintentional leak determined from the acoustic data may be compared to, validated with, and/or combined with an amount of unintentional leak derived from an intentional leak characteristic curve for the respiratory therapy system 120. Combining the amount of unintentional leak based on the acoustic data with an amount of unintentional leak derived from the intentional leak characteristic curve can provide a better estimate, using a Kalman filter approach for instance, of the unintentional leak. Such a combination from different sources of determination provides a better estimate particularly when there is variability in the ambient environment (for example, humidification, temperature) that impacts the determinations from one or both sources. In some implementations, if the unintentional leak determined from the acoustic data is found to be low, a higher weightage/confidence can be applied to the determination of unintentional leak derived from the intentional leak characteristic curve. Subsequently, as the unintentional leak determined from the acoustic data increases, the weightage/confidence data applied to the determination of unintentional leak derived from the intentional leak characteristic curve during this higher leak period may be decreased.

In some implementations, the leak will manifest as a broadband increase in the acoustic spectra representing the collected acoustic data. However, a localized breaking of the seal between the cushion of the user interface 124 and the face of the user 210 may generate a high-intensity whistle-like sound, which can manifest as a sharp peak in the acoustic spectra at the fundamental frequency and subsequent peaks at harmonic frequencies, typically with lower magnitude. This is shown in FIG. 5B, which illustrates the peaks A and B at fundamental frequency and a harmonic frequency, respectively, representing the high-intensity whistle-like sounds on the acoustic spectra. This acoustic pattern can be detected on the acoustic spectra and the magnitude and associated frequencies of the peaks can be used to estimate the amount (e.g., volume of air, expressed as e.g. L/min) of the underlying leak. In practical terms, such patterns may be associated with low levels of unintentional leak but can result in significant discomfort for the user 210 and/or the bed partner 220.

In some implementations, the user 210 can detect occurrence of the leak and indicate to the system 100 that there is leak in the airflow within the respiratory therapy system 120. For that purpose, the user 210 can use the user device 170 (which could be a smart phone, a tablet, a laptop, a smart speaker, etc.) to indicate to the system 100 about the occurrence of the leak. The system 100 can use this user input in the detection of the occurrence of the leak in the respiratory therapy system 120, in addition to the acoustic data, as an alternative to the acoustic data, or as a confirmation of the detection based on the acoustic data.

In some implementations, the occurrence of the leak is detected based at least in part by comparing a current measurement with a previously-obtained baseline measurement. For example, the volume of noise occurring during the operation of the respiratory therapy system 120 can be compared to a baseline volume known to occur when no leak is present. In another example, time-domain or frequency-domain measurements can be compared to baseline time-domain or frequency-domain measurements obtained when it was known that no leak was present in the respiratory therapy system 120. Any acoustic patterns or acoustic signatures analyzed using the acoustic data can be relative or absolute changes compared to baseline acoustic patterns or acoustic signatures obtained when it was known that no leak was present in the respiratory therapy system 120. In some implementations, the acoustic patterns or acoustic signatures can also be normalized or scaled by flow data measured by the flow rate sensor 134 and pressure data measured by the pressure sensor 132.

In some implementations, the location of the leak is determined by comparing current acoustic data with baseline characteristics determined for each of the one or more microphones 140—for example, the baseline characteristics for the microphones 342, 344, 346 described above with respect to FIG. 3B. The comparison of current acoustic data with the baseline characteristics identifies which of the three microphones 342, 344, 346 matches with the changes (e.g., an increase, a decrease, or optionally relative to a pre-determined threshold) observed in the amplitude and/or high-frequency content relative to the baseline characteristic along the path of airflow in the respiratory therapy system 120. Such determination of location of the leak may compensate, as needed, for any speed changes in the blower motor 345 induced by the detected leak.

As another example, in an implementation having two microphones 140 (not shown), a simple comparison of values of acoustic parameters (e.g., amplitude and/or spectral content of the acoustic data) determined from the acoustic data for each of the microphones can be used to determine the location of a leak. In such cases, the relative change in values of acoustic parameters relative to baseline characteristics can indicate that the location of a leak is closer to the microphone that exhibits the largest change.

FIG. 6A illustrates a graphical representation of acoustic power levels over a ˜5.5-minute time period of no leak and a ˜5-minute time period of user interface leak. The acoustic data generated by the one or more microphones 140 detects variability in noise levels and acoustic characteristics or patterns associated with the acoustic signatures corresponding to the ˜5.5-minute time period of no leak and the ˜5-minute time period of leak from the respiratory therapy system 120. As shown in FIG. 6A, a leak from the user interface 124 (mask leak) can be clearly detected from the plotted acoustic data over the time periods, based on sound level features (for example, features capturing variations with time of the parameters associated with the acoustic data, weighted combination of multiple acoustic features and/or patterns) and spectral features such as the acoustic energy ratio in the different frequency bands (between about 0 and about 8 kHz in the plot of FIG. 6A).

FIG. 6B illustrates a comparative graphical representation of flow rate, pressure in the user interface 124, and leak rate over the periods of leak and no leak of FIG. 6A. As indicated by FIG. 6B, the detection of leak in the user interface 124 from the acoustic data of FIG. 6A clearly correlates with an indication of leak in the user interface 124 from the data on pressure, flow rate and leak rate in the user interface 124 over the same ˜5.5-minute time period of no leak and the same ˜5-minute time period of leak in the user interface 124.

FIG. 7A illustrates a graphical representation of acoustic power levels over a time period during which leaks occur in the respiratory therapy system 120 and where the leaks can be classified based on source or location of the leak from the respiratory therapy system 120. The acoustic data generated by the one or more microphones 140 may have acoustic features having different acoustic characteristics depending on the type of leak. Different conditions may have to be satisfied (for example, different thresholds may be applied to the parameters in the acoustic data) depending on the type of leak. As shown in FIG. 7A, a continuous leak from the user interface 124 is indicated by a distinct acoustic signature than a continuous leak from the mouth of the user 210, based on sound level features (for example, features capturing variations with time of the parameters associated with the acoustic data, weighted combination of multiple acoustic features and/or patterns) and spectral features such as the acoustic energy ratio in the different frequency bands (between about 0 and about 8 kHz in the plot of FIG. 7A). The distribution of acoustic energy across the different frequency bands in FIG. 7A illustrates a clear difference between the two types of leaks as indicated by a higher acoustic energy content in the lower frequency bands for the continuous leak in the user interface 124 and in the higher frequency bands for the continuous leak from the mouth of the user 210.

FIG. 7B illustrates a comparative graphical representation of flow rate, pressure in the user interface 124, and leak rate over the time period of FIG. 7A. As indicated by FIG. 7B, the detection of leak in the user interface 124 and the leak from the mouth of the user from the acoustic data of FIG. 7A clearly correlates with corresponding indications of the leak in the user interface 124 and the leak from the mouth of the user 210 from the data on pressure, flow rate and leak rate in the user interface 124 over the same time period of FIG. 7A.

In some implementations, a machine learning model (e.g., Artificial Neural Network, K-Nearest Neighbor Algorithm, Convolutional Neural Network, Support Vector Machine, etc.) may be trained using acoustic data (and/or frequency spectrum of the data, cepstrum of the data, or a combination thereof) collected under various operating conditions, leak sources, and leak volumes. The machine learning model would be trained to identify the location of the leak from acoustic data labelled with, for example, information about the location, type, and/or volume of leak, etc. The trained machine learning model would then determine the location, the type, and/or the volume of the leak upon receiving, as input, raw acoustic data, frequency spectrum of the data, cepstrum of the data, or a combination thereof.

In some implementations, to aid in detection and/or identification of the leak from the acoustic data, the acoustic data received from the microphones is first decomposed into different portions as belonging to different components forming the path of airflow (e.g., the blower motor 345 in the respiratory therapy device 122, the conduit 126, the user interface 124 having the one or more vents 125) using techniques such as blind-source-separation, independent component analysis, singular value decomposition, principle component analysis, empirical mode decomposition or deconvolution, and the like.

In some implementations, the acoustic signature (for example, acoustic energy content in specific high frequency bands) identified from the acoustic data can be used to determine one or more types of the vents 125 in the user interface 124. In such implementations, the one or more types of vents 125 in the user interface 124 may indicate a feature or type of user interface 124 being used such as, but not limited to, a form factor of the user interface 124, a model of the user interface 124, a manufacturer of the user interface 124, or a combination thereof. For example, different designs of the vents 125 may have a characteristic sound such as, but not limited to, a hissing sound (for designs having a series of pinprick holes in the vents 125), a diffused sound of low-frequency (for designs having a mesh diffuser, which reduces turbulence and high-frequency content in the generated acoustic data), etc.

If a leak in airflow within the respiratory therapy system 120 is detected, a variety of different actions can be undertaken. In some implementations, the action can include transmitting a notification to the user 210, and/or to a third party such as, but not limited to, the user's spouse, roommate, family member, healthcare provider, or another person. The notification can include an indication to the user 210 or to the third party that the leak has been detected in the respiratory therapy system 120. The notification can indicate to the user 210 the specific location of the leak from the respiratory therapy system (e.g., the housing 127 of the respiratory therapy device 122, the user interface 124, the conduit 126, etc.), a measure of disturbance caused by the leak, or can generally indicate that leak has been detected. The notification can provide instructions to solve the leak, or recommend the user 210 to use a different type of user interface 124 (for example, use of a user interface 124 that has a chin strap or a full-face masking component if the leak is detected from the mouth of the user 210, use of increased humidification if the mouth of the user 210 becomes dry).

In some implementations, the detected leak, the location of the leak, or the amount of the leak can be used to estimate the comfort level of the user 210 or the bed partner 220, for example, by feeding into a comfort score. The degree to which the leak impacts comfort level may be based on the acoustic intensity of the noise generated by the leak, loudness of the noise, and other characteristics such as, but not limited to, sharpness, roughness, and fluctuation strength of the noise.

In some implementations, the techniques of the method 1000 can implemented when the user 210 is not in a sleep session. For example, when the user 210 is setting up the respiratory therapy system 120 or getting ready for bed but before the sleep session has started (for example, before the user 210 gets into the bed 230 or before the user 210 begins to try to fall asleep), the acoustic data can be generated and analyzed to determine if there is any leak in the user interface 124, the conduit 126, the respiratory therapy device 122 or another components of the respiratory therapy system 120. The user interface 124 may be worn by the user 210 to determine occurrence of a leak anywhere in the respiratory therapy system 120, or otherwise occluded to, for example, determine occurrence of a leak anywhere therein other than at a sealing component of the user interface 124 and the face of the user 210. If the leak is detected, the user 210 can be notified, so that the user 210 can (i) reduce or remove the leak (for example, by adjusting the pressure of the pressurized air from the blower motor 345 in FIG. 3B), (ii) repair or replace the leaking component, (iii) use the user device 170 to indicate to the system 100 that there is a leak in the respiratory therapy system 120, (iv) adjust or change the type of user interface 124.

In other implementations, the techniques of the method 1000 can be implemented during a sleep session while the user 210 is awake. For example, the acoustic data can be generated during the sleep session, and if the analysis of the acoustic data indicates that there is leak from the user interface 124, the conduit 126, the respiratory therapy device 122 or any other portion of the respiratory therapy system 120, the system 100 can send the user 210 a notification such that the user 210 can undertake immediate action to address the leak.

One or more steps of the method 1000 can be implemented using any element or aspect of the system 100 (FIGS. 1-2) described herein. Further, while the method 1000 has been shown and described herein as occurring in a certain order, more generally, the steps of the method 1000 can be performed in any suitable order.

In certain implementations, when the determination of the leak in airflow in the respiratory therapy system 120 indicates there is no, or substantially no, unintentional leak, the flow rate data and pressure data associated with pressurized data delivered to the user 210 during the sleep session is used to determine an intentional leak characteristic curve for the respiratory therapy system 120. As defined herein, “substantially no leak” implies a leak less than 0.1 liters per second and optionally, less than 0.05 liters per second. In some implementations, if the intentional leak determined from the acoustic data is found to be low, a higher weightage/confidence can be applied to the determined intentional leak characteristic curve. Further, if the intentional leak determined from the acoustic data becomes high enough, the amount of intentional leak can be used to offset the flow data used to determine the intentional leak characteristic curve.

As discussed above, in some implementations, the system 100 includes a blower motor 345 (FIG. 3B) in the respiratory therapy device 122 for generating a flow of pressurized air, which is then supplied to an airway of the user 210 for a time period of therapy (e.g., a first one or more sleeping cycles). The flow of pressurized air has a first nominal pressure value. In some implementations, the first nominal pressure value is a pressure that the blower motor 345 attempts to maintain during operation of the respiratory therapy device 122, by modifying one or more operating parameters to at least account for breathing of the user 210. In some such implementations, the one or more operating parameters include a rotation per minute of the blower motor 345, a power supplied to the blower motor 345, or a combination thereof. The first nominal pressure value can be a value not exceeding the predetermined pressure threshold. For example, the first nominal pressure value can be between about 1 cm H₂O to about 8 cm H₂O, such as 1.5 cm H₂O, 2 cm H₂O, 2.5 cm H₂O, 3 cm H₂O, 3.5 cm H₂O, 4 cm H₂O, 4.5 cm H₂O, 5 cm H₂O, 5.5 cm H₂O, 6 cm H₂O, 6.5 cm H₂O, 7 cm H₂O, 7.5 cm H₂O.

Further, as mentioned above, the system 100 can include a flow rate sensor 134 and a pressure sensor 132. The flow rate sensor 134 is configured to generate flow data over the time period of therapy, while the pressure sensor 132 is configured to generate pressure data over time period of therapy during which the CPAP system attempts to maintain the constant predetermined air pressure for the respiratory therapy system 120. For example, FIG. 5A illustrates a portion of such flow data and pressure data associated with the user 210 of the respiratory therapy system 120. In some implementations, the pressure sensor 132 and/or the flow rate sensor 134 can be coupled to or integrated in any component or aspect of the respiratory therapy system 120 described herein, while the microphone 140 can be communicatively coupled to or integrated in any component or aspect of the system 100. In some implementations, the pressure data can include pressure data at the respiratory therapy device 122 measured from a pressure sensor 132 in the respiratory therapy device 122 and/or pressure data at the user interface 124 generated by a pressure sensor (not shown) coupled to the user interface 124 or otherwise determined by the respiratory therapy system 120.

While the respiratory therapy device 122 attempts to maintain the first nominal pressure value, a first plurality of flow rate values is generated using the flow rate sensor 134 and a first plurality of pressure values is generated using the pressure sensor 132 for the first time period of therapy. The first time period of therapy may be (i) at about a beginning of an inhalation portion of a breath of the user 210, (ii) at a minimum flow rate of the breath, (iii) at about a beginning of an exhalation portion of the breath of the user 210, (iv) at a maximum flow rate of the breath, or (v) any combination thereof. A first Cartesian coordinate is determined based on at least one of the first plurality of flow rate values and at least one of the first plurality of pressure values, as describe herein. For example, in some implementations, the first Cartesian coordinate is determined based at least in part on a first flow rate value and a first pressure value. In some implementations, the first Cartesian coordinate is determined based at least in part on an average of the first plurality of flow rate values and an average of the first plurality of pressure values. Alternatively, in some implementations, instead of estimating and/or calculating the first Y value using the first plurality of pressure values, the first nominal pressure value is used as the first Y value.

Once the first Cartesian coordinate is determined, the respiratory therapy device 122 adjusts the flow of pressurized air to a second nominal pressure value for a second time period (e.g., a second one or more breathing cycles). In some implementations, the supplied flow of pressurized air is increased to the second nominal pressure value that is greater than the first nominal pressure value. For example, the second nominal pressure value can be between about 0.1 cm H₂O to about 1 cm H₂O greater than the first nominal pressure value, such as 0.1 cm H₂O, 0.2 cm H₂O, 0.25 cm H₂O, 0.5 cm H₂O, 0.75 cm H₂O, or 1 cm H₂O greater than the first nominal pressure value. Additionally, or alternatively, the second nominal pressure value is between about 0.1% to about 5% greater than the first nominal pressure value, such as 0.1%, 0.15%, 0.2%, 0.25%, 0.3%, 0.35%, 0.4%, 0.45% or 0.5% greater than the first nominal pressure value.

In some implementations, the change between the first nominal pressure value and the second nominal pressure value can be of any suitable value, so long as both the first nominal pressure value and the second nominal pressure value are low enough to cause little unintentional leak. In some implementations, the change between the first nominal pressure value and the second nominal pressure value is small (e.g., between about 0.05 cm H₂O to about 0.1 cm H₂O). However, in some such implementations, a change too small (e.g. <0.05 cm H₂O) may lead to inaccuracy in the resulting parameter computation (e.g., the estimation of the non-zero constants for the intentional leak characteristic curve).

While the respiratory therapy device 122 attempts to maintain the second nominal pressure value, a second plurality of flow rate values for the second time period is generated. In addition, a second plurality of pressure values for the second time period is generated. Subsequently, a second Cartesian coordinate is determined based on at least one of the second plurality of flow rate values and at least one of the second plurality of pressure values, as describe herein. Alternatively, in some implementations, instead of estimating and/or calculating the second Y value using the second plurality of pressure values, the second nominal pressure value is used as the second Y value. Similarly, in some implementations, a third, fourth, fifth, or more Cartesian coordinates can be determined while the respiratory therapy device 122 adjusts the flow of pressurized air.

Receiving the plurality of flow rate values and the corresponding plurality of pressure values over the time period of therapy as inputs, an intentional leak characteristic algorithm can output an intentional leak characteristic curve for the respiratory therapy system 120 associated with the user 210. As discussed herein, in some implementations, an airflow pathway is formed by the respiratory therapy device 122, the user interface 124, and the conduit 126. The conduit 126 creates a first impedance Z1, which in turn causes a pressure drop ΔP that is a function of the total flow rate Qt. The pressure at the user interface 124 Pm is the device pressure Pd less the pressure drop ΔP through the conduit 126, where ΔP(Qt) is the pressure drop characteristic of the conduit 126.

Pm=Pd−ΔP(Qt) (1)

One or more vents 125 of the user interface 124 creates a second impedance Z2. The vent flow Qv is related to the pressure Pm at the user interface 124 via the vent characteristic f:

Pm=f(Qv) (2)

Combining equation (1) with equation (2), the device pressure Pd may be written as

Pd=f(Qv)+ΔP(Qt) (3)

Any unintentional leak, which is unknown and unpredictably variable, may create a third impedance Z3. The fourth impedance Z4, the capacitance Clung, and the variable pressure source Plung represent characteristics of the user 210. Thus, the total flow rate Qt is equal to the sum of the vent flow rate Qv, the leak flow rate Qleak, and the respiratory flow rate Qr:

Qt=Qv+Qleak+Qr (4)

In some implementations, the respiratory flow rate Qr averages to zero over a plurality of respiratory cycles (e.g., breathing cycles), because the average airflow into or out of the lungs must be zero. As such, taking an average of each flow rate over the plurality of respiratory cycles, the vent flow rate may be approximated as:

\tilde{Q}v=\tilde{Q}t−\tilde{Q}leak (5)

The tilde (˜) indicates the average value over the plurality of respiratory cycles. The process of averaging may be implemented by low-pass filtering with a time constant long enough to contain the plurality of respiratory cycles. The time constant can be of any suitable duration, such as five seconds, ten seconds, thirty seconds, one minute, etc. However, other time intervals are also contemplated.

Combining equations (3) and (5), the average device pressure \tilde{P}d may be written as

\tilde{P}d=f(\tilde{Q}t−\tilde{Q}leak)+ΔP(\tilde{Q}t) (6)

Absent any leak flow (e.g., Qleak=0), the average total flow rate \tilde{Q}t may be referred to as the bias flow rate Qb. Equation (6) can then be written to reflect the relationship between bias flow rate Qb and average device pressure \tilde{P}d that characterizes the respiratory therapy system 120:

\tilde{P}d=f(Qb)+ΔP(Qb) (7)

The relationship, which is the intentional leak characteristic curve for the respiratory therapy system 120, is determined by the vent characteristic f(Q) and the conduit pressure drop characteristic ΔP(Q).

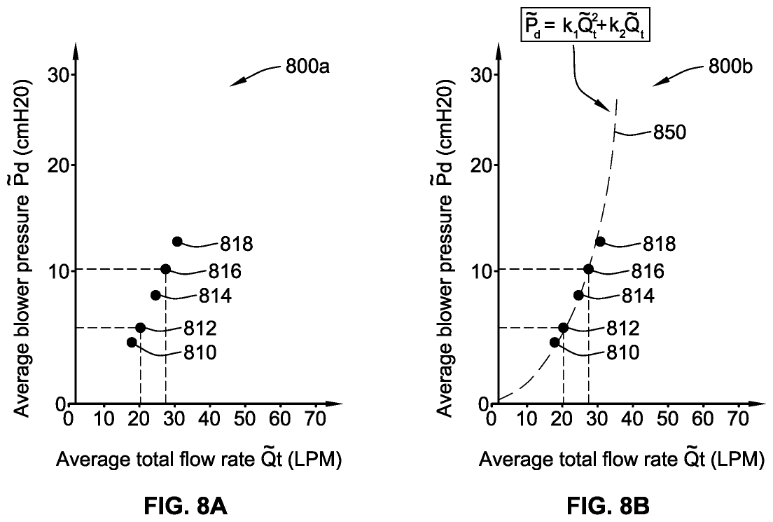

Referring now to FIG. 8A, a scatter plot 800a of average total flow rate \tilde{Q}t (in liters per minute) versus average device pressure \tilde{P}d (in cm H₂O) is depicted. Each Cartesian coordinate includes an X value and a Y value. As shown, five Cartesian coordinates 810, 812, 814, 816, and 818 are plotted in the scatter plot 800a over a time period of therapy. Each Cartesian coordinate may also be expressed as (\tilde{Q}t, \tilde{P}d).

For example, a first Cartesian coordinate 812 has a first X value that is about 20 liters per minute, and a first Y value that is about 6 cm H₂O. As such, the first Cartesian coordinate 812 can be expressed as (20, 6). The first X value can be estimated and/or calculated based at least on a first plurality of flow rate values generated over a first time period. In some implementations, the first X value is an average flow rate value of the first plurality of flow rate values generated over the first time period.

In some implementations, the first time period is a predetermined time interval, such as five seconds, ten seconds, thirty seconds, one minute, two minutes etc. In some implementations, the first time period includes one or more full breathing cycles, such as one breathing cycle, two breathing cycles, five breathing cycles, ten breathing cycles, etc. Therefore, in some implementations, the first X value is the average flow rate value of the first plurality of flow rate values generated over one or more breathing cycles.

Similarly, the first Y value can be estimated and/or calculated based at least on a first plurality of pressure values generated over the first time period. Each of the first plurality of pressure values corresponds to a respective one of the first plurality of flow rate values. For example, in some implementations, each of the first plurality of flow rate values has a corresponding time stamp, using which the respective one of the first plurality of pressure values can be identified. Therefore, in some implementations, the first Y value is the average pressure value of the first plurality of pressure values generated over one or more breathing cycles.

A second Cartesian coordinate 816 has a second X value that is about 28 liters per minute, and a second Y value that is about 10 cm H₂O. As such, the second Cartesian coordinate 816 can be expressed as (28, 10). The second X value and the second Y value of the second Cartesian coordinate 816 can be estimated and/or calculated the same way as, or similar to, the first X value and the first Y value of first Cartesian coordinate 812.

Referring to FIG. 8B, based at least in part on the first Cartesian coordinate 812 and the second Cartesian coordinate 816, an intentional leak characteristic curve 850 can be fitted in the plot 800b. For example, in some implementations, the intentional leak characteristic curve 850 may be approximated using a polynomial equation, such as a quadratic equation:

\tilde{P}d=k₁Q_{b}²+k₂Q_{b} (8)

The parameters of the intentional leak characteristic curve approximated as a quadratic equation are two non-zero constants (or coefficients), k₁ and k₂, which characterize the series concatenation of the vent characteristic f and the air circuit pressure drop characteristic ΔP. In some implementations, the polynomial equation defines an intentional leak of the respiratory therapy system 120 by providing a corresponding flow rate of intentional leak for a given pressure.

If at least two Cartesian coordinates are known, the non-zero constants k₁ and k₂ can be solved. For example, using the first Cartesian coordinate 812 (20, 6) and the second Cartesian coordinate 816 (28, 10), equation (8) can be solved and re-written as approximately:

$${\tilde{P}}_{d} = \frac{1}{140} {Q}_{b}^{2} + \frac{11}{ζ} {Q}_{b} \qquad ( 9 )$$

In other words, the non-zero constant k₁ is 1/140 (about 0.00714), and the non-zero constant k₂ is 11/70 (about 0.157) for the quadratic curve going through the first Cartesian coordinate 812 and the second Cartesian coordinate 816. Therefore, in some implementations, the intentional leak characteristic curve 850 can be estimated and/or defined as equation (9).

In some implementations, the non-zero constants k₁ and k₂ depend on (i) the unit for the pressure values, (ii) the unit for the flow rate values, (iii) the one or more vents 125 in the user interface 124 for the respiratory therapy system 120, (iv) the user interface 124 for the respiratory therapy system 120, or (v) any combination thereof. The non-zero constants k₁ and k₂can also vary with different types of the user interface 124, different manufacturers of the user interface 124, and/or different batches of the user interface 124. For example, with the pressure values measured in cm H₂O and the flow rate values measured in liters per minute, the non-zero constants k₁ and k₂ can be about 1/154 and about 1/7 respectively, for a particular vent 125 of the user interface 124 of the respiratory therapy system 120.

In some implementations, the polynomial equation may have more than two non-zero constants, such as three non-zero constants, four non-zero constants, five non-zero constants, etc. For example, the polynomial equation may be expressed as:

\tilde{P}d=k₁Q_{b}²+k₂Q_{b}+k₃ (10)

In some implementations, the polynomial equation may involve a power of three, four, five, etc. For example, the polynomial equation may be expressed as:

\tilde{P}d=k₁Q_{b}³+k₂Q_{b}²+k₃Q_{b} (11)

In some implementations, unintentional leak is more likely to occur in a respiratory therapy system 120 where the pressure values are high. To get a better fitted intentional leak characteristic curve, only Cartesian coordinates with lower values are used to solve the polynomial equations 8, 9, 10, and 11. For example, in some implementations, the Cartesian coordinates where the Y values do not exceed a predetermined pressure threshold are used. The predetermined pressure threshold may be associated with a low likelihood of unintentional leak, such as less than about 10 cm H₂O, less than about 9 cm H₂O, less than about 8 cm H₂O, less than about 7 cm H₂O, less than about 6 cm H₂O, less than about 5 cm H₂O, less than about 4 cm H₂O, less than about 3 cm H₂O, or less than about 2 cm H₂O.

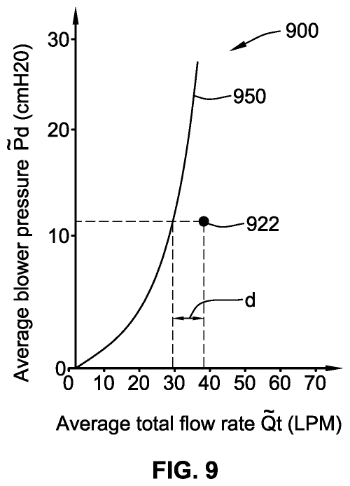

Referring now to FIG. 9, an intentional leak characteristic curve 950 of a respiratory therapy system 120 associated with the user 210 is shown on a plot 900. The intentional leak characteristic curve 950 relates each value of average device pressure \tilde{P}d to the bias flow rate Q_{b} at that value of device pressure. Any excursions, e.g. Cartesian coordinate 922, to the right of the intentional leak characteristic curve 950 is indicative of an unintentional leak, which exhibits as an increased flow rate for a given device pressure.

Over a time period of respiratory therapy, where the average device pressure \tilde{P}d varies, and absent any unintentional leak, the point (\tilde{Q}t, \tilde{P}d) moves up and down, following the intentional leak characteristic curve 950. However, having an unintentional leak can cause the point (\tilde{Q}t, \tilde{P}d) to move to the right of the intentional leak characteristic curve 950 for a time period. If the unintentional leak resolves, the point (\tilde{Q}t, \tilde{P}d) returns to the intentional leak characteristic curve 950. Therefore, the unintentional leak may show up on the plot 900 as excursions to the right of the intentional leak characteristic curve 950.

For example, at a point (shown as Cartesian coordinate 922) where the user experiences an unintentional leak, \tilde{Q}t is greater than the bias flow Qb at that value of average device pressure \tilde{P}d. The difference between the actual \tilde{Q}t and the bias flow Qb at the given average device pressure \tilde{P}d is the distance d, which is about 9 liters per minute, as shown in FIG. 9. On the other hand, excursions to the left of the intentional leak characteristic curve 950, such as the points (\tilde{Q}t, \tilde{P}d) where \tilde{Q}t is less than the bias flow Qb, may indicate vent blockages (e.g. as a result of head movement relative to the pillow on which the user 210 is sleeping and which causes blockage of vent(s) 125 by the pillow).

As discussed herein, a respiratory therapy system 120 typically includes components such as the respiratory therapy device 122, the conduit 126, and the user interface 124. A variety of different forms of user interface 124 may be used with a given respiratory therapy device 122, such as nasal pillows, nasal prongs, a nasal mask, a nose and mouth (orinasal) mask, or a full face mask. Furthermore, different lengths and diameters of conduits 126 may be used. In order to provide improved control of therapy delivered to the user interface 124, it may be advantageous to estimate treatment parameters such as the pressure in the user interface 124, the vent flow rate, and the unintentional flow rate. In respiratory therapy systems using estimation of treatment parameters, knowledge of the type of component being used by the user 210 can enhance the accuracy of treatment parameter estimation, and therefore the efficacy of therapy.

To obtain knowledge of component type, some respiratory devices 122 include a menu system that allows the user 210 to enter and/or select the type of system components, including the user interface 124 being used (e.g., brand, manufacturer, form, model, serial number, mask family, size etc.). Once the types of the components are entered and/or selected by the user 210, the respiratory therapy device 122 can select appropriate operating parameters of the blower motor 345 (FIG. 3B) that best coordinate with the selected components, and can more accurately monitor treatment parameters during therapy. However, in some instances, the user 210 may not select the type of component correctly, or at all, leaving the respiratory therapy device 122 in error or ignorant about the type of component in use.

As such, in some implementations, the intentional leak characteristic algorithm described herein can be used to identify the user interface 124. For example, if the conduit pressure drop characteristic ΔP is known, (e.g., because the type of conduit 126 is known, or through a prior calibration operation), then the parameters of the intentional leak characteristic curve effectively characterize the vent 125, which in turn is indicative of the type of user interface 124.

In some implementations, the user interface 124 may be identified by comparing the computed non-zero constants k₁ and k₂ to a data structure such as an array or database having pairs (k₁, k₂) associated with known types of the user interface 124, when used with the known conduit 126. The type of user interface 124 associated with the stored pair (k₁, k₂) that most closely matches the computed non-zero constants k₁ and k₂ may be taken as the type of the user interface 124.

Alternatively, the pressure drop ΔP(\tilde{Q}t) may be subtracted from each value of the average device pressure \tilde{P}d before fitting the quadratic equation to the resulting intentional leak characteristic curve of the user interface 124. The resulting non-zero constants k₁ and k₂ may then be compared to a data structure of pairs (k₁, k₂) associated with known types of the user interface 124 to identify the user interface 124 or access data for operations of the respiratory therapy device 122 that is associated with use of particular user interfaces 124.

Thus, the detected parameters can be compared to the expected parameters over a period of time, collecting longitudinal data and cross-sectional data. In some implementations, the respiratory therapy system 120 may be able to determine production variation by understanding a batch of masks used as the user interface 124, and use this for production quality improvement.

In some implementations, the respiratory therapy system 120 can check for variation over time, and understand if a seal of the user interface 124 is degrading over time (as the respiratory therapy system 120 can determine how long a specific user interface 124 has been in use based on an RFID tag and/or user input), and what are the conditions that are giving rise to unintentional leak (e.g., is it position dependent, has it changed based on recommendation to tighten or loosen headgear, has the seal followed an expected degradation cycle (assuming regular washing), is it showing accelerated wear, etc.).

In some implementations, the user interface 124 being used can be determined by: user input, detecting the user interface 124 optically, detecting the user interface 124 via RFID, detecting the user interface 124 using electronics (e.g., a user interface detection component), detecting the conduit 126 via a connector of a heated tube that has electronics (e.g., electronic connection connecting to the control system 110 of the respiratory therapy device 122, an electronic chip, etc.), or any combination thereof. Thus an initial curve can be selected that describes a correctly functioning new user interface of this type. The initial curve may be specific to the respiratory therapy device 122, the operating mode, the operating parameters, the user interface 124 (e.g., brand, manufacturer, form, model, serial number, mask family, size etc.), or any combination thereof.

In some implementations, over time, the respiratory therapy system 120 may also be able to select models of expected behavior of partially worn or fully worn out user interfaces 124 of this type as the initial curve, such as from a look up table, or from a cloud system. These expected models may describe different levels of occlusion in the vent 125, the conduit 126 (such as for user interfaces 124 that have airflow through soft tubing around the head) and different levels of seal wear, headgear stretching, and so forth. Thus, by selecting an appropriate initial model, the respiratory therapy system 120 can detect intentional leak and unintentional leak through a sleep session, and/or across multiple sleep sessions.

In some implementations, the respiratory therapy system 120 can detect low intentional leak (e.g., lower than expected intentional leak), early indications of blockage in the vent 125 (e.g., due to excess moisture, ageing and so forth), and/or poor sleep position of the user 210 (such as when the vent 125 is occluding). In some such implementations, prompts can be sent to the user 210 (i) to alert the user 210 of these issues, (ii) to recommend repair or replacement of the user interface 124 and/or the vent 125, or (iii) both.

In some implementations, the respiratory therapy system 120 can detect bed pillows, or other object such as a user's arm, blocking the exhalation ports (such as the one or more vents 125). In some such implementations, prompts can be sent to the user 210 to suggest a change of bedding, a change in configuration or types of the user interface 124, a change in body position, or any combination thereof.

Further, in some implementations, unintentional leak can also occur around the humidification tank 129. This can manifest as a high pitched sound (audible and potentially annoying to some people with higher frequency hearing, such the bed partner 220 of the user 210), as well as another source of “intentional leak” that is not associated with the airflow in the vent 125. In order to avoid confusing with the unintentional leak, the respiratory therapy system 120 may also process acoustic sounds to check if the acoustic signature of such a leak in the humidification tank 129 is present. The respiratory therapy system 120 may recommend to the user 210 to re-seat the humidification tank 129. By separating this source of leak, a more accurate separation of leak type (and suggested resolution) can be provided to the user 210 and/or health care professional.

Having a more robust and/or accurate model of leak reporting will result in more users remaining as users, e.g., these users use might otherwise have discontinued use as a result of inconsistent and/or inaccurate leak reporting. In some implementations, the present disclosure may provide systems and methods for determining if a mouth leak is occurring, which allows a recommendation to the user for a full face mask, before the user quits therapy from problems with nasal masks and the resulting mouth leak.

In some implementations, selecting the type setting of the user interface 124 on the respiratory therapy device 122 is not necessary. The present disclosure provides an automatic system of detecting the type of user interface 124 by estimating the intentional leak characteristic curve, and comparing the estimated intentional leak characteristic curve to existing data for the particular type of the user interface 124 (e.g., from a lookup table, from other users of the same type of the user interface 124, from previous data associated with the same user 210, or any combination thereof).

According to some implementations of the present disclosure, alternative or additional methods can be used to determine an intentional leak characteristic curve for a respiratory therapy system 120. These methods improve the impedance determination of the user interface 124 and/or the conduit 126. Such impedance determination can in some implementations be characterized by the intentional leak characteristic curve. Benefits of improved impedance determination include: (i) improving leak estimation accuracy, (ii) resolving under-reporting of leak in the user interface 124, (iii) providing impedance feature input to assist the identification of the user interface 124, (iv) providing diffuser loss feature input to assist the estimation and/or determination of mouth leak, (v) exploiting air circuit signals and/or impedance.

One or more elements or aspects or steps, or any portion(s) thereof, from one or more of any of claims 1 to 60 below can be combined with one or more elements or aspects or steps, or any portion(s) thereof, from one or more of any of the other claims 1 to 60 or combinations thereof, to form one or more additional implementations and/or claims of the present disclosure.

While the present disclosure has been described with reference to one or more particular embodiments or implementations, those skilled in the art will recognize that many changes may be made thereto without departing from the spirit and scope of the present disclosure. Each of these implementations and obvious variations thereof is contemplated as falling within the spirit and scope of the present disclosure. It is also contemplated that additional implementations according to aspects of the present disclosure may combine any number of features from any of the implementations described herein.

1. A method comprising:
receiving acoustic data associated with airflow caused by operation of a respiratory therapy system during a sleep session of a user;
analyzing at least a portion of the acoustic data to determine a value of a parameter associated with the at least a portion of the acoustic data; and
determining an occurrence of a leak during the sleep session in response to the determined value of the parameter satisfying a condition.

2. The method of claim 1, wherein the determining the occurrence of the leak includes determining a leak, a type of leak, an amount of leak, or any combination thereof.

3. The method of claim 1, wherein the acoustic data is generated by, and received from, a plurality of microphones communicatively coupled to the respiratory therapy system, and further comprising:
determining, for each of the plurality of microphones, a baseline audio characteristic, wherein each of the baseline audio characteristics is unique to a respective one of the microphones,
wherein the analyzing the at least a portion of the acoustic data includes analyzing the at least a portion of the acoustic data from each of the plurality of microphones with respect to each of the baseline audio characteristics.

4. The method of claim 3, wherein at least one microphone of the plurality of microphones is (i) coupled externally to a conduit of the respiratory therapy system, (ii) positioned at least partially within a respiratory therapy device of the respiratory therapy system, (iii) coupled externally to a user interface of the respiratory therapy system, (iv) coupled directly or indirectly to a headgear associated with the user interface, (v) coupled to a mobile device that is communicatively coupled to the respiratory therapy system, (vi) electrically connected with a. circuit board of a respiratory therapy device within the respiratory therapy system, (vii) in acoustic communication with the airflow in the respiratory therapy system, (viii) configured to be in direct fluid communication with the airflow, (ix) or (x) any combination thereof.

5. The method of claim 3, wherein the microphone is positioned at least partially outside of a housing of a respiratory therapy device of the respiratory therapy system, the microphone being at least partially movable relative to the housing of the respiratory therapy device to aid in directing the microphone towards the user.

6.-9. (canceled)

10. The method of claim 1, wherein the parameter includes acoustic intensity, acoustic volume, acoustic frequency, acoustic energy ratio, or any combination thereof.

11. The method of claim 1, wherein the satisfying the condition includes exceeding a threshold value, not exceeding the threshold value, staying within a predetermined range of values, staying outside the predetermined range of values, or any combination thereof.

12. The method of claim 1, wherein the determining the occurrence of the leak includes determining an intentional leak that is indicative of airflow venting from one or more vents associated with the respiratory therapy system.

13.-28. (canceled)

29. The method of claim 1, wherein the analyzing the at least a portion of the acoustic data includes:
generating a frequency spectrum from the acoustic data; and
identifying one or more features of the frequency spectrum that are indicative of presence of the leak.

30. The method of claim 1, wherein the analyzing the at least a portion of the acoustic data includes:
generating a mel-frequency cepstrum from the at least a portion of the acoustic data; and
determining one or more mel-frequency cepstral coefficients from the mel-frequency cepstrum,
wherein the leak is detected based at least in part on the one or more mel-frequency cepstral coefficients.

31.-39. (canceled)

40. A system comprising:
a respiratory therapy device configured to generate a flow of pressurized air;
a user interface configured to aid in delivery of the flow of pressurized air to a user;
a conduit configured to connect the respiratory therapy device and the user interface;
a microphone;
a memory storing machine-readable instructions; and
a control system including one or more processors configured to execute the machine-readable instructions to:
generate, using the microphone, acoustic data associated with the flow of the pressurized air during a sleep session of the user;
analyze at least a portion of the generated acoustic data to determine a value of a parameter associated with the generated acoustic data; and
determine an occurrence of a leak during the sleep session in response to the determined value of the parameter satisfying a condition.

41. The system of claim 40, wherein the microphone is (i) coupled externally to a conduit of the respiratory therapy system, (ii) positioned at least partially within a respiratory therapy device of the respiratory therapy system, (iii) coupled externally to a user interface of the respiratory therapy system, (iv) coupled directly or indirectly to a headgear associated with the user interface, (v) coupled to a mobile device that is communicatively coupled to the respiratory therapy system, (vi) electrically connected with a. circuit board of a respiratory therapy device within the respiratory therapy system, (vii) in acoustic communication with the airflow in the respiratory therapy system, (viii) configured to be in direct fluid communication with the airflow, (ix) or (x) any combination thereof.

42. The system of claim 40, wherein the microphone is positioned at least partially outside of a housing of a respiratory therapy device of the respiratory therapy system, the microphone being at least partially movable relative to the housing of the respiratory therapy device to aid in directing the microphone towards the user.

43.-46. (canceled)

47. system of claim 40, wherein the parameter includes acoustic intensity, acoustic volume, acoustic frequency, acoustic energy ratio, or any combination thereof.

48. The system of claim 40, wherein the satisfying the condition includes exceeding a threshold value, not exceeding the threshold value, staying within a predetermined range of values, staying outside the predetermined range of values, or any combination thereof.

49. The system of claim 40, wherein the control system is further configured to execute the machine-readable instructions to determine the presence or absence of the leak, a type of the leak, an amount of the leak, or any combination thereof.

50.-53. (canceled)

54. The system of claim 40, wherein the control system is further configured to execute the machine-readable instructions to analyze the at least a portion of the acoustic data by:
generating a frequency spectrum from the acoustic data; and
identifying one or more features of the frequency spectrum that are indicative of presence of the leak.

55. The system of claim 40, wherein the control system is further configured to execute the machine-readable instructions to analyze the at least a portion of the acoustic data by:
generating a mel-frequency cepstrum from the at least a portion of the acoustic data; and
determining one or more mel-frequency cepstral coefficients from the mel-frequency cepstrum,
wherein the leak is detected based at least in part on the one or more mel-frequency cepstral coefficients.

56. The system of claim 55, wherein the control system is further configured to execute the machine-readable instructions to analyze the at least a portion of the acoustic data by inputting the one or more mel-frequency cepstral coefficients into a machine learning model configured to detect the leak based at least on values of the one or more mel-frequency cepstral coefficients.

57.-60. (canceled)

61. The system of claim 40, wherein the microphone includes a plurality of microphones, wherein the control system is further configured to:
determine, for each of the plurality of microphones, a baseline audio characteristic, wherein each of the baseline audio characteristics is unique to a respective one of the microphones,
wherein the control system analyzes the at least a portion of the generated acoustic data by analyzing the at least a portion of the acoustic data from each of the plurality of microphones with respect to each of the baseline audio characteristics.
